(19) United States
(12) Patent Application Publication
(10) Pub. No.: US 2026/0306020 A1
(43) Pub. Date: Oct. 8, 2026

(54) WEARABLE ELECTRONIC DEVICES AND METHODS FOR DETECTING EMOTIONAL STATES AND PROVIDING ACTIONABLE NEUROFEEDBACK

(71) Applicant: AWEAR TECHNOLOGIES INC., San Francisco, CA (US)

(72) Inventors: Antonio FORENZA, San Francisco, CA (US); Tommaso BARTALUCCI, San Francisco, CA (US)

(21) Appl. No.: 19/485,429

(22) PCT Filed: May 14, 2024
(86) PCT No.: PCT/US2024/029332

§ 371 Date: Nov. 18, 2025

Related U.S. Application Data
(60) Provisional application No. 63/503,073, filed on May 18, 2023.

(51) Int. Cl. A61B 5/00; A61B 5/16; A61B 5/374; G09B 5/06

(52) U.S. Cl. CPC A61B 5/6803; A61B 5/0022; A61B 5/165; A61B 5/374; A61B 5/742; A61B 5/7475; G09B 5/065

(57) ABSTRACT

A wearable electronic device, such as a hat or glasses, and methods for detecting emotional states and providing actionable neurofeedback to promote mental wellness. The device is equipped with different types of sensors including electroencephalogram (EEG) electrodes, functional near-infrared spectroscopy (fNIRS), photoplethysmography (PPG), electrodermal activity (EDA), or skin or body temperature sensors that measure biological parameters or brain signals and feed them back to a hardware platform and a software platform. The hardware and software platforms process and analyze that data and determine different emotional states of the user via AI/ML classifiers. Results of the analysis are presented over an application that includes UX/UI for providing neurofeedback to the user. The neurofeedback is provided in terms of actionable metrics or training programs designed to improve mental wellness.

### FIELD OF THE INVENTION

The described embodiments relate generally to a wearable electronic devices and methods to promote mental wellness. The device is a hat, or headband, or glasses, or earbuds, or other wearable electronic devices designed for everyday use and equipped with sensors that analyze and interpret brain or body signals to provide users with real-time feedback on their emotional state. The methods provide users with real-time neurofeedback to bring awareness, correlate their emotional states to daily life events, enable neurofeedback-based therapy, or neurological regulation. The methods also include mental wellness programs to enhance mental resilience and balance.

### BACKGROUND OF THE INVENTION

According to the American Psychological Association, more than a quarter of US adults reported that stress severely affects their daily functions and health, causing fatigue, anxiety and depression [1]. Fatigue costs US employers $136B annually in lost productivity [2]. The present example implementations discloses the first neurotech electronic device that is truly wearable for everyday use and raises awareness of our emotional states to enhance our emotional resilience.

The global market of wearable tech devices was valued at about US$100B this year [3]. Over 25% of US adults use health and fitness wearables today (e.g. Apple Watch, Fitbit, Whoop, Oura, etc.) [4-8]. While those devices track our physical wellness, they are unable to provide a reliable measure of our mental wellness. One of the first commercial wearable products intended to measure moods or emotions was the so called “mood ring” that came out in the 1970s and used an analog technology consisting of a thermochromic element (i.e., the “mood stone”) that changed color due to change in body temperature. That product was based on the property that our moods affect our body temperature through the Autonomic Nervous System (ANS). However, there are many other factors that affect the ANS and also change our body temperature, so that product was unable to provide a direct mapping of the moods to the colors of the mood stone. Other products today use digital technology including temperature sensors to measure body or skin temperature for identifying strain and recovery, emerging illness, and phases of the menstrual cycle [177-179], but they are unable to measure or classify our emotional states. Another popular digital technology used in today's wearable electronic devices is photoplethysmography (PPG) to measure Heart Rate (HR) or Heart Rate Variability (HRV). HRV provides an indirect indication of physical stress resulting from activity of the ANS, which is also triggered by many other physiological factors such as fatigue, sleep, diet, dehydration, sickness, etc. Therefore, HRV alone does not provide a reliable measure of physical stress, let alone mental stress or any other information about our emotional states. Some of the wearables (e.g., Fitbit Sense 2, Empatica, Feel, Nowatch, Happy Ring [9-13]) combine HRV with Electrodermal Activity (EDA) to get an indirect indication of mental stress from ANS activity. EDA, however, can only estimate the intensity of emotions without distinguishing positive from negative valence (i.e., it cannot tell whether the user is aroused because of stress or joy). Further, wristbands or rings using HRV/EDA suffer from measurement delays and artifacts due to hand's motion or sweat.

Unlike related art, the present example implementations disclose a wearable neurotech electronic device and methods that use different types of sensors or combinations of different types of sensors to provide a direct measure of biomarker or brain signals generated by neural activities underlying our emotional states. For example, one embodiment of the example implementations uses electroencephalogram (EEG) electrodes that provide actionable neurofeedback of a wide range of emotional states including valence and arousal, unlike related art HRV/EDA-based wearables. Another embodiment uses functional near-infrared spectroscopy (fNIRS) sensors to determine emotional states by measuring brain maps or active regions of the brain. Another embodiment combines EEG, fNIRS, PPG, HRV, EDA and skin or body temperature information to provide prediction of emotional states.

The market segment of neurotech devices is currently growing at a CAGR of 12% and is expected to reach over US$20B by 2026 [14]. Over 441 million smart headbands were sold worldwide as of 2021 [15] and between 200-500 million people practice mindfulness globally [16]. There are over 50 neurotech wearable devices currently in the market that provide neurofeedback based on EEG, electromyography (EMG) or fNIRS technologies (e.g., Muse by InteraXon, FocusCalm by Brainco, Mendi, Epoc by Emotiv, NextMind, CTRL-Labs, etc. [17-74]). These wearables have different form factors such as: clinical caps, headbands, wristbands, headphones, earbuds or helmets. None of these products, however, are suitable for being worn in everyday life (e.g., during sports, at the gym, for outdoor activities, at social events, or while shopping) because they look more like lab equipment than true wearables. Also, they only address limited use cases such as enhancing focus/meditation or detecting fatigue, but they do not provide any information about emotional states. Their sales volume has been rather limited, for example, InteraXon has sold only 50K units to date since 2014 [74] or Brainco only 20K units within the first three years of operation [75]. By contrast, currently over 40 million Apple Watches are sold annually [76].

The wearable electronic device and methods disclosed in the present example implementations taps into the market segment of neurotech devices to address the need for people to enrich our mindfulness practice for building emotional resilience, reducing stress and cultivating happiness everyday. Unlike related art neurotech wearable devices, the present example implementations measures brain signals through the wearable electronic device and maps them into emotional states through the disclosed methods that provide actionable neurofeedback.

Research on classification of emotions or affect started in the field of psychology with the seminal work by Russell on the circumplex model of affect [77-79]. Later on, from 2003-2023, emotional classification research moved into the field of neuroscience with several studies on measuring and classifying emotions through EEG and functional magnetic resonance imaging (fMRI) [80-153]. The present example implementations leverages the advances on emotional classification by the research in psychology and neuroscience to disclose novel wearable electric devices and methods for detecting emotions and providing actionable neurofeedback. We project sales volume of the wearable electronic device disclosed in the present example implementations will be much higher than existing neurotech devices because of its wider typology of consumers due to its everyday portability and larger set of applications which are at the core of the innovation of the present example implementations.

### SUMMARY OF THE INVENTION

Embodiments of the system, devices, methods and apparatuses described in the present disclosure are directed to a wearable electronic device having a set of sensors that may be used to sense and determine brain signals or biological parameters of a user that wears it. The sensors may include for example EEG, EMG, fMRI, fNIRS, Magnetoencephalography (MEG), positron emission tomography (PET), event-related optical signal (EROS), electrocardiogram (ECG or EKG), Photoplethysmography (PPG), electrodermal activity (EDA) [154-163], or sensors for sweat analysis, or different combinations of the above sensors. One embodiment uses any type of antennas (e.g., dipoles, patch antennas, microstrip antennas, ferrite rod antennas or any radio frequency (RF) antenna used in wireless communications) that receive or transmit electromagnetic fields (EMF), since brainwaves are indeed electromagnetic waves generated by charged particles as a result of neural activities in the brain and as such they are modeled by the same Maxwell equations as EMF [164-169].

There are different types of brainwaves characterized by different frequencies and the most common ones are Delta waves (less than 4 Hz), Theta waves (4-8 Hz), Alpha waves (8-12 Hz), Beta waves (12-30 Hz) and Gamma waves (greater than 30 Hz). The Delta waves are usually associated with deep sleep. The Theta waves are associated to REM sleep and states of meditation. The Alpha waves are recorded during wakeful relaxation with closed eyes, are reduced with open eyes and sleep, while they are enhanced during drowsiness, and have a variant called Mu wave. The Beta waves are associated to waking consciousness and are split into three sections including Low Beta Waves (12.5-16 Hz, “Beta 1”); Beta Waves (16.5-20 Hz, “Beta 2”); and High Beta Waves (20.5-28 Hz, “Beta 3”). The Gamma waves can range up to 140 Hz with the 40 Hz point being of particular interest, they are correlated with working memory, attention and perceptual grouping.

One embodiment of the example implementations takes the form of a hat such as baseball cap, beanie, or any other type or style of hat. Another embodiment uses different fashionable designs that users feel comfortable and proud to wear in everyday life. The methods are implemented over one or a plurality of consumer devices (e.g., smartphones, smartwatches, etc.) and cloud-native platforms that collect and analyze data from many users and extract brainwave fingerprints through the power of AI/ML methods. Metrics are presented to the users via e.g., iOS/Android app to bring awareness of our emotional states, correlate them to daily life events and enhance our emotional resilience. For example, the app can train users to maintain an optimal level of hormetic stress while preventing burnouts, depression or chronic stress [170].

There are multiple application for the present example implementations, including but not limited to: mental training, medical diagnosis, or commercial applications. Some use cases for mental training are: monitoring level of focus, concentration, workload, engagement, or fatigue; building emotional resilience (e.g., for athletes, or corporate executives); enhancing emotional and mental awareness (e.g., label emotional states throughout the day, measure emotions, rumination or microstess); acting training (e.g., Meisner technique and Method acting train actors to experience truthful emotions on set). Further, the example implementations can also be used for medical purposes such as: detecting emotions of infants, patients with neurodegenerative disease, Deaf people; detecting upcoming seizures for people affected by epilepsy; diagnosing strokes, dementia, alcholism, different neurological disorders such as Alzheimer's disease, autism, schizophrenia, major depressive disorder (MDD), chronic pain, post-traumatic stress disorder (PTSD), attention deficit hyperactivity disorder (ADHD), attention deficit disorder (ADD), autism, rumination, or others. Finally, there are multiple commercial applications for the present example implementations, including but not limited to: marketing research, or consumer research, or neuroscience research by leveraging cloud-data collected from multiple users; detecting fatigue for track drivers, airline or general aviation pilots, air traffic controllers, train operators, etc.; augmented reality (AR) or virtual reality (VR) applications (e.g., detecting eye movement with EEG, detecting emotions created by different AR/VR experiences); human activity recognition (HAR) to detect e.g., muscular activities, emotional control, motor planning, emotional expression, reading activities.

### BRIEF DESCRIPTION OF DRAWINGS

FIG. 1 shows a functional diagram of the system architecture including the wearable electronic device being the hat (hardware platform), the software platform and the network connecting the two;

FIG. 2 shows a functional diagram of the system architecture including the wearable electronic device being the glasses (hardware platform), the software platform and the network connecting the two;

FIGS. 3A and 3B show the location of the sensors over the baseball cap;

FIGS. 4A and 4B show the location of the sensors inside the baseball cap;

FIG. 5 shows one of the standard maps of EEG electrode locations (related art);

FIG. 6 shows the method that uses an array of EEG electrodes on the hat to generate a volume of EMF energy within the brain;

FIG. 7 shows an example method of generating volumes of EMF energy within the brain or determining the spatial location of the sources of specific brainwaves or brain signals;

FIG. 8 shows the location of the sensors over the beanie hat;

FIG. 9 shows the location of the sensors over the glasses;

FIG. 10 shows the block diagram of the hardware platform including sensors and control unit (CU);

FIGS. 11A-D show the placement of the CU at different location of the hat;

FIG. 12 shows the CU one exemplary installation to the hat;

FIGS. 13A-B show different exemplary implementations of the display at the CU;

FIG. 14 shows different components of the software platform and networks;

FIGS. 15A-B shows two different graphs of the circumplex model (related art);

FIGS. 16A-C show different pages of the UX/UI for the application that provides neurofeedback;

FIG. 17 shows direct communication between to different wearable electronic devices;

FIG. 18 shows the action potential propagating between two neighbor neurons and the volume of EMF energy created at different neuron locations with the array of EEG electrodes;

FIG. 19 shows the method to generate the volume of focused energy 1801 within the brain.

### DETAILED DESCRIPTION

The present example implementations relate generally to a wearable electronic device, and more particularly to one or a plurality of wearable electronic devices and methods to detect emotional states and provide actionable neurofeedback to enhance mental wellness. The example implementations provide a wearable electronic device equipped with one or a plurality of different types of sensors including but not limited to EEG, fNIRS, ECG, PPG, EDA, or temperature sensors that overcome one or more of the problems or limitations of the related art and provides real-time feedback on physical or mental wellness, including different type of emotional states. Unlike related art, the present example implementations are not limited to detecting body responses based on HRV or EDA as result of ANS activities, rather it comprises additional sensors such as fNIRS sensors or EEG electrodes that provide neurofeedback based on brain signals. As such, the present example implementations provide more accurate biological parameter to measure mental wellness than related art. Additionally, it provides neurofeedback of wide range of emotional states including valence and arousal, unlike related art technology based on HRV or EDA that only provides information about arousal and cannot distinguish between e.g., stress and joy. Further, unlike related art neurotech devices implemented as clinical caps, headbands, wristbands, headphones, earbuds or helmets, the present example implementations disclose novel form factors for wearable electronic devices that measure brain signals such as hats or glasses. The example implementations may be implemented in various embodiments, and the detailed description below provides exemplary embodiments and accompanying figures for a complete understanding of the example implementations.

### System Architecture

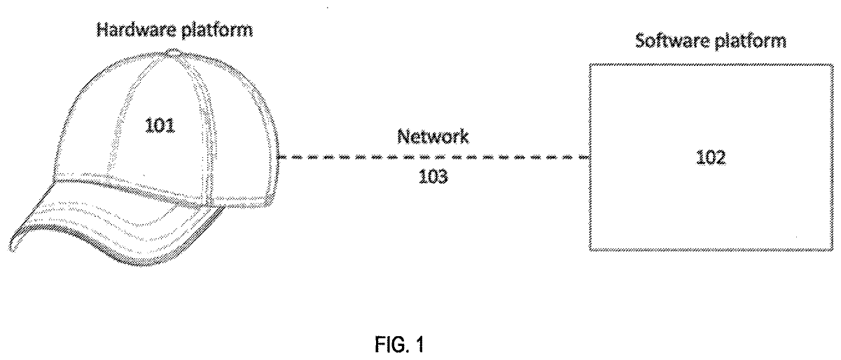

The present example implementations include three or more components as shown in FIG. 1, including: a hardware platform 101, a software platform 102 and a network 103. The hardware platform comprises of a wearable electronic device 101 that has different form factor such as a hat, or glasses, or earbuds, or hair clips, or hair band, or headband, or safety helmet, or safety cap, or motorcycle helmet, or racing helmet, or skiing helmet, or climbing hat, or by cycle bump cap, or any other wearable that is worn on someone's head. In one exemplary embodiment of the example implementations, the wearable electronic device is a beanie, or a baseball cap, or a cloche, or a fez, or a bucket, or a beret, or a ivy cap, or a Breton, or a newsboy cap, or a visor, or a trapper, or a turban, or a panama hat, or a cowboy hat, or a cartwheel, or a sombrero, or a fedora, or a floppy, or a boater, or a homburg, or a bowler, or a trilby, or a top or a fascinator, or any other type or style of hats. When the wearable electronic device is implemented as a baseball cap, it is designed in one or a plurality of different types including but not limited to: classic baseball cap, snapback cap, fitted cap, trucker cap, flexfit cap, dad hat, 5-panel cap, 6-panel cap, 7-panel cap, camper cap, bucket hat, curved brim cap, flat brim cap, beanie cap with brim, running cap, visor cap, mesh cap, performance cap, golf cap, military cap.

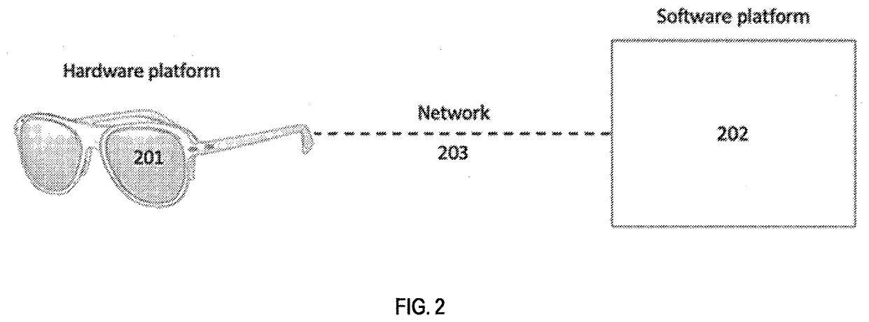

In another exemplary embodiment of the example implementations shown in FIG. 2, the wearable electronic device is one or a plurality of the following types of glasses 201: single vision glasses, bifocal glasses, trifocal glasses, progressive glasses, reading glasses, computer glasses, safety glasses, sports glasses, polarized sunglasses, mirrored sunglasses, photochromic sunglasses, gradient sunglasses, clip-on sunglasses, fitover sunglasses, blue light blocking glasses, driving glasses, fashion glasses, anti-fatigue glasses, anti-glare glasses, night vision glasses, or any other type of glasses. The glasses are of different shapes including but not limited to Round glasses, oval glasses, square glasses, rectangular glasses, cat-eye glasses, butterfly glasses, wayfarer glasses, aviator glasses, clubmaster glasses, geometric glasses, wraparound glasses, rimless glasses, semi-rimless glasses, browline glasses, oversized glasses, or any other type of glasses.

In another exemplary embodiment of the example implementations, the wearable electronic device is one or a plurality of the following types of hair clips: Alligator clip, Barrette, Banana clip, Bobby pin, Butterfly clip, Claw clip, Crocodile clip, Duckbill clip, Snap clip, French clip.

The software platform 102 and 202 comprises an application running on a device or in the cloud, or partially on a device and partially on the cloud. In one embodiment of the example implementations, the application runs on any of the Apple operating systems (OS) including but not limited to macOS, iOS, iPadOS, watchOS, tvOS, HomePod Software, AudioOS, iPod Software. In another embodiment of the example implementations, the application runs on any of the Android operating systems including but not limited to: Android OS, Android Wear OS, Android TV OS, Android Auto OS, Android Things OS, Fire OS, Oxygen OS, One UI, MIUI, EMUI. In another embodiment, the application runs on any type of smartphone or smartwatch devices by any brand included but not limted to: Apple, Samsung, Huawei, Xiaomi, Oppo, Vivo, OnePlus, Google, LG, Sony, HTC, Motorola, Nokia, Asus, Lenovo, ZTE, Meizu, BlackBerry, Alcatel, TCL. In one embodiment, the application is running on one or a plurality of smartphone devices, including but not limited to: Apple iphone 13 series (iPhone 13, iPhone 13 mini, iPhone 13 Pro, iPhone 13 Pro Max), Samsung Galaxy S21 series (Galaxy S21, Galaxy S21+, Galaxy S21 Ultra), Google Pixel 6 and Pixel 6 Pro, Xiaomi Mi 11 series (Mi 11, Mi 11 Pro, Mi 11 Ultra), OnePlus 9 and OnePlus 9 Pro, Oppo Find X3 series (Find X3 Pro, Find X3 Neo, Find X3 Lite), Vivo X60 series (X60, X60 Pro, X60 Pro+), Motorola Edge 20 series (Edge 20, Edge 20 Pro, Edge 20 Lite), Nokia X20 and X10, Sony Xperia 1 III and Xperia 5 III. In one embodiment, the application is running on one or a plurality of smartwatch devices, including but not limited to: Apple Watch Series 1-7, Samsung Galaxy Watch 4 and Watch 4 Classic, Fitbit Versa 1-3, Sense and Sense 2, Garmin Venu 2 and Venu 2S, Fossil Gen 5E and Gen 6, TicWatch Pro 3, Amazfit GTS 2 and GTR 2, Huawei Watch GT 2 Pro, Oppo Watch 2.

In another embodiment of the example implementations, the application runs on any type of cloud, including but not limited to: far-edge cloud, edge-cloud, Public cloud, Private cloud, Hybrid cloud, Community cloud, Distributed cloud, Multi-cloud, Inter-cloud, Fog/cloud edge, Serverless cloud. In another embodiment the application runs on any cloud by different companies including but not limited to: Amazon Web Services (AWS)—Amazon Elastic Compute Cloud (EC2), Amazon Simple Storage Service (S3), Amazon Relational Database Service (RDS), Microsoft Azure—Azure Virtual Machines, Azure Blob Storage, Azure SQL Database, Google Cloud Platform (GCP)—Compute Engine, Cloud Storage, Cloud SQL, IBM Cloud—Virtual Servers, Object Storage, Databases for MongoDB, Oracle Cloud—Compute, Storage, Database, Alibaba Cloud—Elastic Compute Service (ECS), Object Storage Service (OSS), Relational Database Service (RDS).

The network 103 and 203 is one or a plurality of wireless networks including but not limited to: Wi-Fi, Bluetooth, NFC, Zigbee, Z-Wave, RFID, Cellular networks (e.g., 2G GSM, 3G WCDMA, HSDPA, 4G LTE, 5G NR, or any 3GPP network), Satellite networks. In another embodiment of the example implementations, the network is one or a plurality of wireline networks including but not limited to: DSL, Cable modem, Fiber optic, T1/E1, T3/E3, SONET/SDH, ISDN, Ethernet.

### Hardware Platform

The hardware platform consists of one or a plurality of wearable electronic devices such as a baseball cap 101 or glasses 201. The wearable electronic devices are equipped with one or a plurality of sensors including but not limited to EEG electrodes, EMG, fMRI, fNIRS, MEG, PET, EROS, ECG or EKG, PPG, EDA, or antennas that receive or transmit EMF, or 3-axis gyroscope, or accelerometer, or global positioning system (GPS) receiver, or barometer, or body or skin temperature, or sensors for sweat analysis, or proximity sensor, or ambient light sensor, or any sensor to identify the ID of the person based on different characteristics of the skin, scalp, hair (including but not limited to shape, consistency, color, sweat level, etc.). The sensors disclosed in the present example implementations have any type of shapes, including but not limited to regular shapes (e.g., Triangle, Square, Rectangle, Pentagon, Hexagon, Heptagon, Octagon, Nonagon, Decagon), or irregular shapes (e.g., Circle, Oval, Heart, Star, Crescent, Trapezoid, Pentagon, Hexagon, Octagon, Rhombus, Parallelogram, Crescent), or any other shape. The shapes are either one, two or three dimensional. In the figures of the present example implementations, the sensors depicted with dashed contour indicate they are placed in the background, behind the object represented in the figure, whereas the sensors depicted with solid contour indicate they are placed in the foreground, on top of the object represented in the figure.

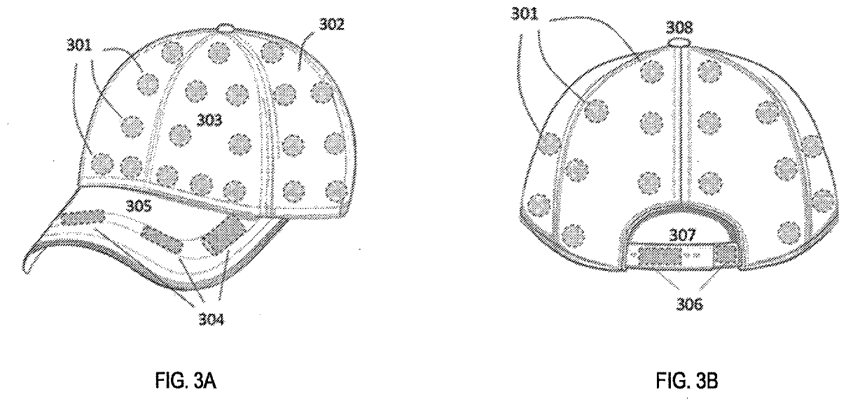

The sensors are placed in one or a plurality of locations of the hat shown in FIG. 3A-B. Note that FIG. 3A-B shows only one type of hat in the form of a baseball cap, but the same example implementations apply to any type, style, shape, color of hats as disclosed above. In one exemplary embodiment of the example implementations, the sensors 301 placed on the front, side or back panels 302 of the crown 303. In another embodiment of the example implementations, the sensors 304 are placed on the visor 305 of the hat. In another embodiment of the example implementations, the sensors 306 are placed on the closure 307 of the hat. The closure is of different types including but not limited to: Adjustable strapback, Snapback, Fitted, Flexfit, Stretch fit, Buckle strap, Velcro strap, Leather strap. In another embodiment, the sensors are placed on the button 308 of the hat. Note that FIG. 3A-B shows the sensors are placed behind the cloth of the hat, and in a different embodiment of the example implementations the same sensors are placed on top of the cloth or integrated within the cloth of the hat, such as textile electrodes (textrodes).

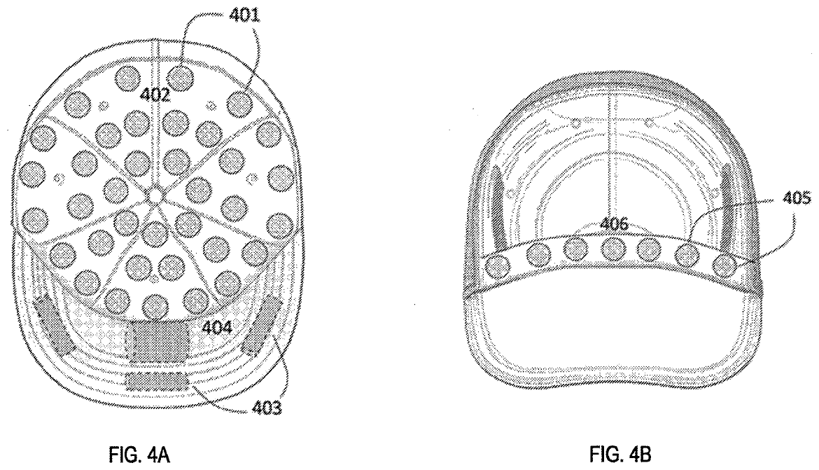

FIG. 4A-B show the bottom and inside views of the hat in FIG. 3A-B. In one embodiment of the example implementations, the sensors are placed on the front, side or back panels of the crown 402. In another embodiment of the example implementations, the sensors 403 are placed on the visor 404 of the hat. In another embodiment of the example implementations, the sensors 405 are placed on the sweatband 406 of the hat or they are integrated with the material of the sweatband, such as textrodes. In another embodiment, the sweatband 406 is a removable headband that can be installed (e.g., via Velcro, touch fastener, clips, buttons, or any other means) to any other hat, or helmet, or cap. In another embodiment of the example implementations, all the sensors in FIG. 3A-B and FIG. 4A-B are integrated to the cloth or material of different components of the hat, such as textrodes.

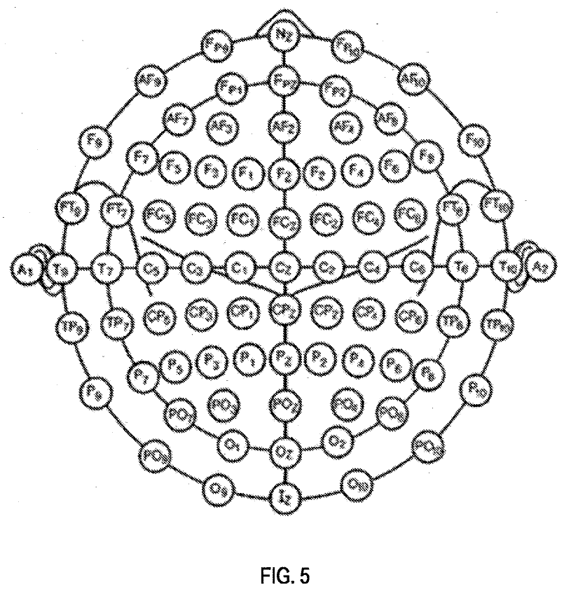

The sensors 301, 304, 306, 401, 403 or 405 are of the same time or different type, including but not limited to EEG electrodes, EMG, fMRI, fNIRS, MEG, PET, EROS, ECG or EKG, PPG, EDA, or antennas that receive or transmit EMF, or different combinations of these sensors. In one exemplary embodiment of the example implementations, the sensors are EEG electrodes placed according to the EEG electrode positions in the standardized 10-20 system, or 10-10 system, or International Federation of Clinical Neurophysiology (IFCN) system shown in FIG. 5, or corresponding to different respective Brodmann areas, or any other position defined by any standardized electrode system with 25, 32, 64, 256 or any number of electrodes [171]. In another embodiment, the electrodes are placed in any position not necessarily reflecting the positions defined in any of the standardized EEG electrode systems. In one embodiment of the example implementations, only a subset of EEG electrodes is used. For example, in [133] it was shown that only a subset of EEG electrodes is needed to detect valence or arousal of different emotions. In one exemplary embodiment of the example implementations, the wearable electronic device uses sensor positions Fp1, Fp2, F3 and F4 to detect the valence dimension of emotional states, whereas sensor positions P3 and P4 to detect the arousal dimension of emotional states. In another embodiment, one or a plurality of additional sensors are used as ground or reference sensors.

The EEG electrodes are of the same type or mixed and matched in different types including but not limited to: Ag/AgCl electrodes, Sintered Ag/AgCl electrodes, Carbon electrodes, Gold electrodes, Tin electrodes, Cup electrodes, Needle electrodes, Comb electrodes, Thread electrodes, Microelectrode arrays (MEAs), Dry electrodes, Wet electrodes, Active or Passive electrodes, conductive-rubber electrodes, conductive carbon-filled rubber electrodes, graphite electrodes. In another embodiment, the EEG electrodes are integrated with any type of soft or spongy material or fabric to reduce or eliminate artifacts from motions of the person wearing the hat (e.g., textrodes), including but not limited to: springs, silicone or any type of polymer, Foam rubber, Polyurethane foam, Memory foam, Neoprene foam, Open-cell foam, Closed-cell foam, Latex foam, Sponge rubber, Cellulose sponge, Cotton, Wool, Silk, Cashmere, Velvet, Chenille, Flannel, Fleece, Microfiber, Jersey, Satin, Suede, Leather, Fur, Faux fur. The EEG electrodes are in direct contact with the skin, forehead or scalp, or penetrate through the hair to reach the scalp, or have no contact whatsoever and are able to receive EMF radiated by charged particles inside the brain and through the scalp.

In one embodiment, the EEG electrode are only used to receive brainwaves or brain signals. In another embodiment, the electrodes are antennas or sensors that transmit EMF through the scalp and into the brain, or implement one of a plurality of different types of transcranial stimulation, including but not limited to: transcranial magnetic stimulation (TMS) or transcranial electric stimulation (TES), Transcranial Direct Current Stimulation (tDCS), High-Definition Transcranial Direct Current Stimulation (HD-tDCS), Transcranial Alternating Current Stimulation (tACS), Transcranial Random Noise Stimulation (tRNS), Electroconvulsive Therapy (ECT), Deep Brain Stimulation (DBS).

One of the advantages of EEG technology over fMRI or fNIRS is its very high temporal resolution. EEG, however, suffers from limited spatial resolution, whereas fMRI or fNIRS technologies are popular for their high spatial resolution. One embodiment of the example implementations comprises a combination of EEG and fMRI/fNIRS technology to provide both high temporal resolution (through EEG) and high spatial resolution (through fMRI/fNIRS). For example, in one of the embodiments, part of the sensors in FIGS. 3-4 are EEG electrodes that provide high temporal resolution, and another part of the sensors are fMRI or fNIRS sensors that provide high spatial resolution. By combining information from these two sets of sensors, it is possible to generate brain maps with high spatial resolution (through fMRI/fNIRS) and measure different brainwaves associated to different regions of the brain with high temporal resolution (through EEG).

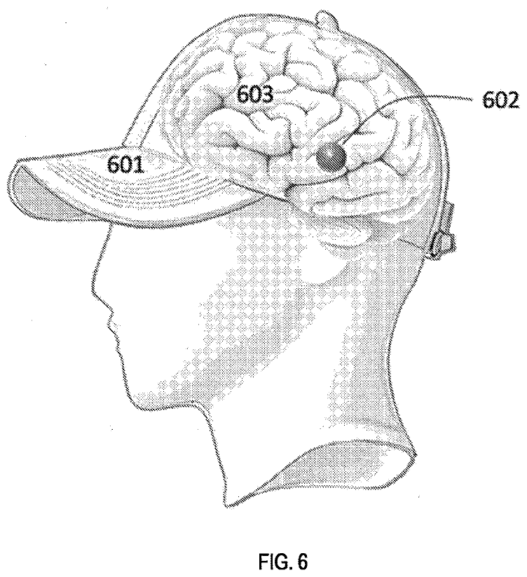

Another embodiment comprises a system and method to increase spatial resolution of EEG alone as shown in FIG. 6. In one exemplary embodiment of the example implementations, the array of EEG electrodes is placed on the hat 601 (e.g., according to the layout in FIGS. 3-4) and used to create one or a plurality of concurrent volumes in space 602 of EMF or brainwave energy within the brain 603. The volume 602 is of any size, e.g., as small as the size or one neuron or even smaller, or as large as the size of the whole brain. In another embodiment, the size of the volume 602 is inverse proportional to the number of EEG electrodes integrated to the hat 601. In one embodiment, 10s, or 100s, or 1000s or more EEG electrodes are integrated into the hat 601 to reduce the size of the volume 602 for increasing the spatial resolution of the array of EEG electrodes. In one embodiment, the EEG electrodes are passive or active sensors that only receive brainwaves or brain signals from one or a plurality of volumes of the brain 602 where multiple neurons exert action potential together. For example, all neurons inside the volume 602 of the brain generate the same type of brainwave, either delta, or theta, or alpha, or mu, or beta, or gamma wave. In another embodiment, the neurons within volume 602 generate different types of brainwaves.

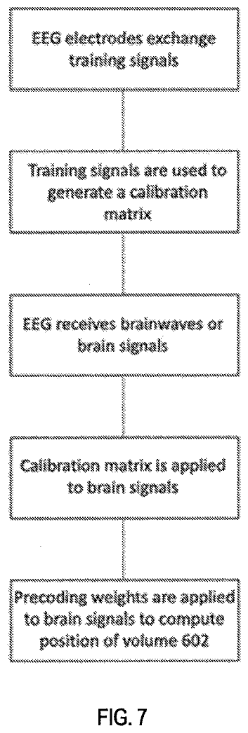

One method consists of multiple steps disclosed in FIG. 7, including but not limited to: i) the EEG electrodes are calibrated by sending or receiving multiple training signals from/to one or a plurality of reference EEG electrodes or antennas; ii) the training signals are used to generate a calibration matrix used to calibrate the EEG electrodes; iii) the brain signals or brainwaves are received from the volume 602 by one or a plurality of EEG electrodes; iv) the calibration matrix is applied to the vector containing the brain signals from all the EEG electrodes, for example via vector-matrix multiplication; v) precoding weights are applied to the vector of brain signal to identify the spatial position of the volume 602. In another embodiment the EEG electrodes are replaced with fNIRS sensors. In a different embodiment, both EEG electrodes and fNIRS sensors are used in combination. The same method is applied to any volume within the brain such that the system and methods identify different locations within the brain where different neurons exert action potential together to generate one or a plurality of brainwaves. For example, the present method identifies the location of the volume 602 in any of the regions of the brain 603, including but not limited to: Amygdala, Brainstem, Cerebellum, Cerebral Cortex, Corpus Callosum, Hippocampus, Hypothalamus, Medulla Oblongata, Pituitary Gland, Prefrontal Cortex, Thalamus.

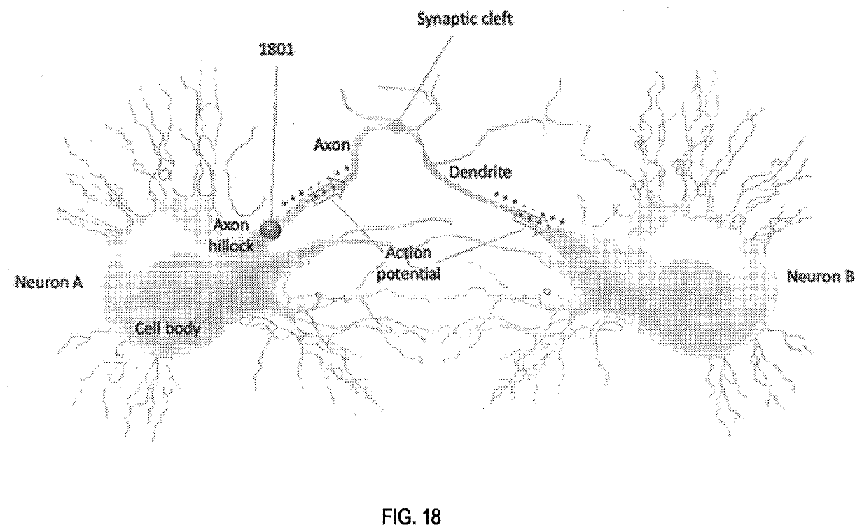

In one embodiment, the system and methods comprise of the array of EEG electrodes in FIG. 3A-B or FIG. 4A-B, or any array of EEG electrodes from any of the existing commercial products [17-74]. The array of electrodes is used to determine the point in space within the brain where the brainwaves or brain signals originate from. To understand how this method works, it is necessary to provide a brief description of the electrochemical phenomena that occur within the brain between millions of neurons that lead to the generation of brainwaves, as depicted in FIG. 18. Every neuron A has as many as 15,000 connections with neighboring neurons B, and transports its information via a nerve impulse called action potential. When sensory receptors (found in various parts of the body, such as the skin, eyes, ears, nose, tongue or internal organs) detect a stimulus (such as light, sound, touch, smell or chemical signals) they generate electrical signals called receptor potentials or graded potentials. These receptor potentials are then transmitted to sensory neurons, which carry the information towards the central nervous system, where further processing occurs. In the central nervous system, these sensory signals are integrated, modified, and ultimately lead to the generation of action potentials. The action potential originates at the axon hillock (the point where the axon leaves the cell body) and propagates through the axon to the synaptic cleft where it causes the release of neurotransmitters (which are transported from the cell body through the axon to the synaptic cleft by the process called axonal transport). The synaptic cleft is a gap less than 40 nm wide between the axon of the transmitting neuron A and the dendrite of the receiving neuron B, where neurotransmitters are released from neuron A to neuron B in a process called synapse. There are over 100 different types of neurotransmitters that carry messages with three possible actions: excitatory (to pass message along to the neighbor neuron), inhibitory (to prevent the message from being passed along any further) and modulatory (to adjust how neurons communicate at the synapse). The most common neurotransmitters are: acetylcholine, dopamine, norepinephrine, epinephrine, histamine, serotonin, glutamate, gamma-aminobutyric acid (GABA), glycine, adenosine, endocannabinoids, nitric oxide, neuropeptides, substance P, endorphins, enkephalins, dynorphins, somatostatin, neuropeptide Y, cholecystokinin, neurotensin, orexin/hypocretin, vasopressin, oxytocin, melanin-concentrating hormone, hypocretin/orexin. Following the synapse, the action potential continues to travel from the synaptic cleft through the dendrite to reach cell body of the neighbor neuron B.

The action potential is produced by voltage-gated ion channels within the cell membrane of the neurons. The cell membrane separates and protects the interior of the neuron characterized by a negative charge from the exterior of the neuron with a positive charge. This difference in voltage (or electrical potential) called membrane potential (typically −70 mV at rest) is due to differences in the concentration of ions (or charged particles) between the inside and the outside of the neuron. The ions at the interior of the neuron are mainly potassium (K⁺) or hydrogen (H⁺), whereas at the exterior are mainly sodium (Na⁺) and chloride (Cl⁻) or calcium (Ca²⁺). These ions transition between the interior and the exterior of the neuron via voltage-gated ion channels activated by changes in the electrical membrane potential near the channel. For example, as the membrane potential is increased, the sodium (Na⁺) ion channels open allowing sodium to move from the exterior to the interior of the neuron, followed by the opening of the potassium (K⁺) ion channel that allows the potassium to move from the interior to the exterior of the neuron. If the membrane potential exceeds a certain value (typically 15 mV higher than the resting voltage of −70 mV), the sodium flow dominates, thereby opening even more sodium (Na⁺) ion channels in a positive feedback cycle (Hodgkin cycle) that proceeds explosively such that the neuron “fires” and produces the action potential.

The frequency of the action potential is referred to as “firing rate” and can be as high as 100 Hz (i.e., upper limit of the spectrum of the brainwaves). The firing rate of a neuron is a complex interplay of excitatory and inhibitory inputs, membrane properties, modulation by neuromodulators, and adaptive mechanisms. Generally speaking, the firing rate of a neuron is determined by the balance between excitatory and inhibitory inputs it receives. For example, high concentration of excitatory neurotransmitters (e.g., glutamate, acetylcholine, dopamine, histamine, epinephrine, norepinephrine) in the synaptic cleft leads to more likely reach the threshold for firing action potentials, leading to a higher firing rate (i.e., generating brainwaves at higher frequencies such as Beta and Gamma waves). By contrast, high concentration of inhibitory neurotransmitters (e.g., serotonin, GABA, glycine) can hyperpolarize the neuron and make it less likely to fire action potentials, resulting in a lower firing rate (i.e., generating brainwaves at lower frequencies such as Delta, Theta and Alpha waves). The superimposition of action potentials from over 50,000 neurons [172] with uniformly oriented axons or dendrites produces the brainwaves measured at the EEG electrodes placed on the scalp (with characteristic magnitude of the order of ~200 μV at a typical distance of 10 cm) [165].

The action potential transmits information through its amplitude, duration and phase. Sodium-based (Na⁺) action potentials usually last for under 1 msec, whereas calcium-based (Ca²⁺) action potentials may last for 100 msec or longer. Since the current produced by the voltage-gated ion channels is significantly larger than the initial current that stimulates the action potential (due to the positive feedback cycle or Hodgkin cycle), the amplitude, duration and phase of the action potential are determined largely by the properties of the excitable membrane, and are independent on the characteristics of the stimulus (e.g., larger currents do not create larger action potentials). Further, excitable parts of the neurons include the axon and cell body, although studies have shown that the most excitable part is the axon hillock.

The methods disclosed hereafter leverage these properties of the action potential for receiving brainwaves selectively from specific regions of the brain or for transmitting focused EMF energy to specific regions of the brain to trigger action potentials.

In one embodiment of the example implementations, the brainwaves measured at the EEG electrodes on the scalp are processed to determine the exact location of the volume 602 in FIG. 6 within the brain 603 where action potentials are produced by neurons firing simultaneously. Note that these methods are different than related art methods for quantitative EEG (qEEG) [184-185], or brain electrical activity mapping (BEAM) [186], or low-resolution brain electromagnetic tomography (LORETA) or standardized LORETA (sLORETA) [187-190]. In fact, related art qEEG or LORETA methods provide electromagnetic tomography only at low resolution and only for the cortex region of the brain. By contrast, the methods in the present example implementations provide high resolution tomography and brain maps for any region inside the brain, even deep inside the brain (i.e., not limited to the cortex as related art methods) as disclosed in the brain cross-section 603 in FIG. 6. In one embodiment, the method detects brainwaves generated by action potentials in a volume 1801 as small as the cell body, or axon hillock, or axon, or synaptic cleft, or dendrite, or any other part of the neuron that exhibits action potential with specific amplitude, duration and phase. One embodiment of the example implementations, comprises one or any combination of different high-resolution or super-resolution techniques for direction of arrival (DOA) estimation commonly used in wireless communications and acoustics, including but not limited to: Multiple Signal Classification (MUSIC) [192], Estimation of Signal Parameters via Rotational Invariance Techniques (ESPRIT) [193], Root-MUSIC (Root-MUltiple Signal Classification), time-reversal signal processing or time-reversal mirror (TRM) for focusing high-energy waves to a point in space, Matrix Pencil Method (MPM), Capon's Minimum Variance Distortionless Response (MVDR), Beam-Space Adaptive Processing (BSAP), Focused Beamformer (FBF), Steered Response Power (SRP), artificial neural network (ANN), Radial Basis Function (RBF) neural network, support vector regression (SVR), support vector machine (SVM), multilayer perceptron (MLP) neural network, deep learning (DL), deep neural network (DNN) [194], or any DOA estimation technique in 2-dimentional space, 3-dimentional space, or any spaces of any dimension.

Further, in some related art methods, the EEG electrodes measure brain signals independently and signal processing techniques are used to generate the “brain maps” to map different frequency bands (e.g., delta, theta, alpha, beta, or gamma waves) into different regions of the brain. By contrast, in the methods of the present example implementations, the EEG electrodes operate cooperatively by combining the brain signals measured by every individual EEG electrode with precoding or postcoding weights to increase the spatial resolution and depth of the brain maps. As a result, the present methods provide for example information about what region of the brain generates specific type of brainwaves at a much greater spatial resolution than any related art methods using qEEG, or BEAM, or LORETA, or sLORETA.

Another embodiment comprises similar methods as in FIG. 7 for transmitting EMF and focusing EMF or brainwave energy to specific regions of the brain (e.g., for transcranial stimulation). In one exemplary embodiment of the example implementations, the method is used to focus EMF energy in one specific volume 1801 of the brain in FIG. 18 to perform any type of transcranial stimulation, including but not limited to TMS, TES, tDCS, HD-tDCS, tACS, tRNS, ECT, or DBS with much higher spatial resolution than systems and methods in related art. In another embodiment, the method is used to stimulate one specific neuron, or neurotransmitter, or synapses, or groups of neurons, or groups of neurotransmitters, or groups of synapses, with one or multiple frequencies and to stimulate the brain to perform a given function. In another embodiment, the method is used to focus EMF energy into a volume 1801 located at the cell body, or axon hillock, or axon, or synaptic cleft, or dendrite, or any other location of the neuron to produce action potential with specific amplitude, duration and phase. Note that, unlike related art methods [173], the methods disclosed in the present example implementations does not require any physical surgery, or any implantable brain-computer interfaces (BCI), or any invasive method to implant any ultra-think probe inside the brain through any neurosurgical robot. In fact, the present method is completely wireless and non-invasive, and can provide the same benefit as related art methods but without implanting probes inside the brain. In another embodiment, the present method is used to cure any disease, or tumors, or any sever condition inside the brain.

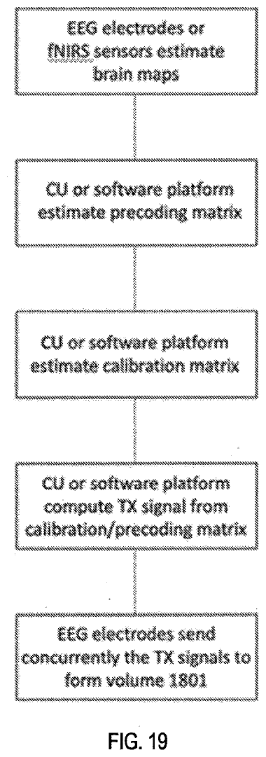

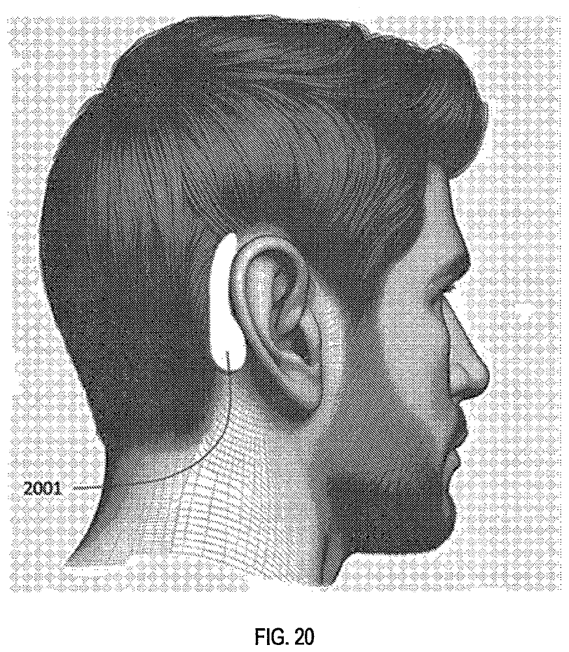

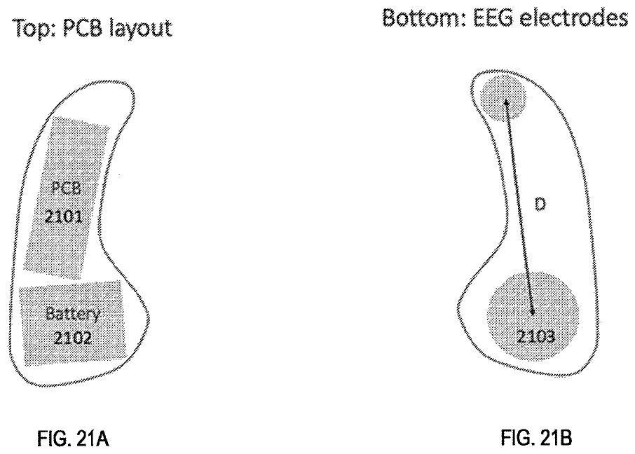

One exemplary method includes the steps disclosed in FIG. 19: i) one or a plurality of EEG electrodes, or fNIRS sensors, or fMRI estimate the brain maps associated to one or a plurality of specific thoughts, emotional states or physical activities. The estimation is carried our via fMRI/fNIRS signal processing or via any of the EEG methods above including qEEG, BEAM, LORETA, sLORETA, MUSIC, ESPRIT, Root-MUSIC, MPM, MVDR, BSAP, FBF, SRP, ANN, RBF, SVR, SVM, MLP, DL, DNN; ii) CU or software platform estimate precoding matrix based on information derived from the brain maps; ili) CU or software platform estimate calibration matrix between all EEG electrodes or fNIRS sensors; iv) CU or software platform compute one or a plurality of transmit (TX) signals by combining the calibration matrix and precoding matrix with one or a plurality of digital signals; v) EEG electrodes or fNIRS sensors send concurrently the TX signals to form the volume 1801 inside the brain. The volume 1801 is used to trigger action potential with specific amplitude, duration and phase at the cell body, or axon hillock, or axon, or synaptic cleft, or dendrite, or any other location of the neuron to generate one or a plurality of specific thoughts, emotional states or physical activities.

One exemplary embodiment of the example implementations uses high-resolution methods in FIGS. 6-7 to detect when the default mode network (DMN) is activated. The DMN consists of multiple regions of the brain including: medial prefrontal cortex, posterior cingulate cortex/precuneus and angular gyrus [195-196]. The DMN is activated when a person is in a state of wakeful rest, such as daydreaming or mind-wandering, and is not focused on the outside world. DMN has been studied with EEG and fMRI to produce brain maps and identify brainwave activities in different regions of the brain [197-200]. In one embodiment of the example implementations, the high-resolution methods disclosed above are used to enable brain mapping and detect DMN activities. In one embodiment, detection of activity by the DMN is used to diagnose different neurological disorders such as Alzheimer's disease, autism, schizophrenia, major depressive disorder (MDD), chronic pain, post-traumatic stress disorder (PTSD), attention deficit hyperactivity disorder (ADHD), attention deficit disorder (ADD), autism, rumination, or others.

In a different embodiment, the sensors 301, 304, 306, 401, 403 or 405 are PPG or EDA sensors. For example, related art [174] shows that the highest density of sweat glands is on the forehead, cheeks, palms and fingers. In one embodiment of the example implementations the sensors 405 are EDA sensors integrated to the sweatband 406 (e.g., textrodes) that measure the galvanic skin response of the forehead. For example, the EDA sensor 405 is used to measure the phasic skin conductance response (SCR) and the tonic skin conductance level (SCL) at the forehead to compute biological parameters that indicate the level of arousal of the person wearing the hat. In another embodiment, this arousal level from the EDA sensor is combined with the information about brain signals from EEG electrodes to determine different levels of arousal and valence characterizing the emotional states of the individual wearing the hat. In a different embodiment, the sensors 405 are PPG sensors used to compute the heart rate (HR), resting heart rate (RHR), Heart rate variability (HRV), Blood oxygen saturation (SpO2), Respiratory rate, Blood pressure, Cardiac output, Stroke volume, Arterial stiffness, or Vascular resistance. In another embodiment, different combinations or EEG, EDA, PPG, or skin or body temperature sensors are installed on the sweatband 406 or any other location throughout the hat to measure brainwaves, brain signals and different biological parameters for detecting and classifying emotional states of the person wearing the hat, including but not limited to levels of arousal or levels of valence.

In a different embodiment, the sensors 301, 304, 306, 401, 403 or 405 are 3-axis gyroscope, or accelerometer, or global positioning system (GPS) receiver, or barometer, or proximity sensor, or ambient light sensor, or skin or body temperature sensors, or sensors for sweat analysis, or any ID sensor. In one exemplary embodiment of the example implementations, the accelerometer is used to estimate different brain-signal characteristics caused by movements of the person wearing the hat. That information is used to remove brain-signal artifacts due to motion and provide clean brain signals in time, frequency and space domains that is used to map brainwaves to different emotional states. In another embodiment, the GPS receiver is used to map the location of the users and determine how certain events cause different emotional states in the users wearing the hat. For example, it is possible to use the GPS information or geo-location of all the users wearing the hat while physically located in the same venue (e.g., outdoor stadium) to infer how the event hosted in that venue (e.g., concert) affects the mood and emotional states of the people attending that event (e.g., for marketing research, or neuroscience research). The GPS receiver is also used to find the hat 101 through the network 103 and the software platform 102 in case the hat is stolen or lost. In another embodiment, the ID sensors or other types of sensors are used to uniquely identify the user and owner of the hat for turning it on/off or lock/unlock the wearable electronic device for security reasons, so no one else can use the hat or access its confidential data when the hat is stolen or lost. This feature is also used to automatically turn on the wearable electronic device when the hat is worn and turn it off automatically when the hat is removed from the person's head to save battery life.

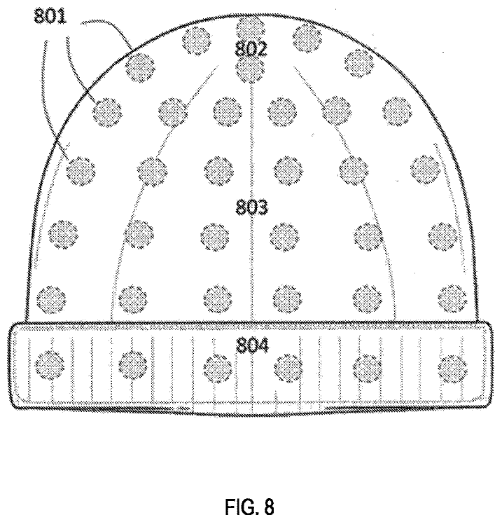

In other embodiments of the example implementations, the wearable electronic device is a beanie hat as shown in FIG. 8. The sensors 801 are integrated inside the crown 802, or the body 803, or the brim 804, or any combinations of those. Like to the baseball cap in FIG. 3A, the sensors of the beanie hat in FIG. 8 are one or a combination of sensors including but not limited to EEG electrodes, EMG, fMRI, fNIRS, MEG, PET, EROS, ECG or EKG, PPG, EDA, or antennas that receive or transmit EMF. The same methods described above for the baseball cap do apply for the beanie hat as well. The same methods described above for the baseball cap do apply for the beanie hat as well.

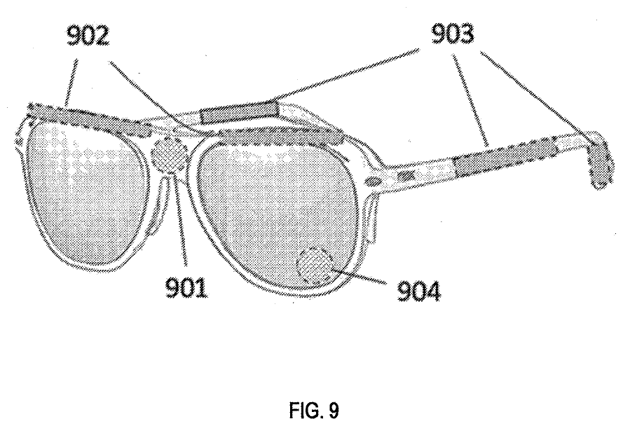

In other embodiments of the example implementations, the wearable electronic devices are glasses as shown in FIG. 9. In one embodiment, the sensors are integrated inside the bridge 901, inside the rim 902, inside the temples 903, or inside the lenses 904. These sensors make direct contact to the skin, or penetrate trough hair via e.g., comb sensors, or do make any contact with the person wearing the glasses and receive brain signals or other biological signals wirelessly. Like to the hats in FIGS. 3-4, 8, the sensors of the glasses in FIG. 9 are one or a combination of sensors including but not limited to EEG electrodes, EMG, fMRI, fNIRS, MEG, PET, EROS, ECG or EKG, PPG, EDA, or antennas that receive or transmit EMF. The same methods described above for the hats do apply for the glasses as well.

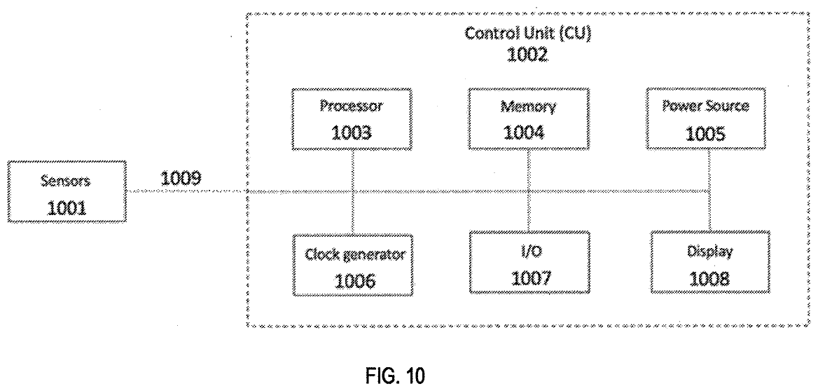

FIG. 10 shows a sample block diagram of a wearable electronic device. In one embodiment, the wearable electronic device is any of the devices disclosed above and in FIGS. 1-9. The devices comprise of one or a plurality of sensors 1001 and one or a plurality of control units (CU) 1002. The sensors 1001 are placed on different locations of the wearable device as shown in FIGS. 3A, 3B, 4A, 4B, 8 and 9, and include but are not limited to: EEG electrodes, EMG, fMRI, fNIRS, MEG, PET, EROS, ECG or EKG, PPG, EDA, or antennas that receive or transmit EMF, or 3-axis gyroscope, or accelerometer, or global positioning system (GPS) receiver, or barometer, or body or skin temperature, or proximity sensor, or ambient light sensor, or any ID sensors. In one embodiment, the sensors are passive or active and comprise power amplifiers or low-noise amplifiers. The CU includes one or a plurality of units including but not limited to: processor 1003, memory 1004, power source 1005, clock generator 1006, input/output (I/O) mechanism 1007 (e.g., I/O device, interface, or port), display 1008. The CU is connected to one or a plurality of sensors via the connection 1009. The connection 1009 includes but is not limited to: Copper wire, Aluminum wire, Silver wire, Gold wire, Nickel wire, Steel wire, Iron wire, Tungsten wire, Platinum wire, Titanium wire, Coaxial cable, Twisted pair cable, Fiber optic cable, Ribbon cable, Shielded cable, Multi-conductor cable, Flat cable, High voltage cable, Low voltage cable.

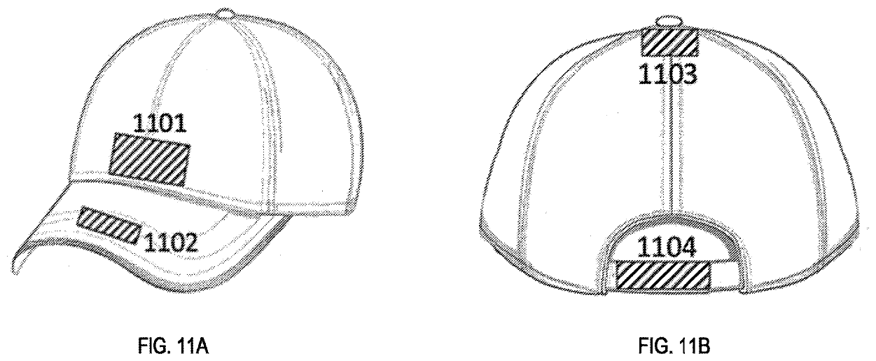

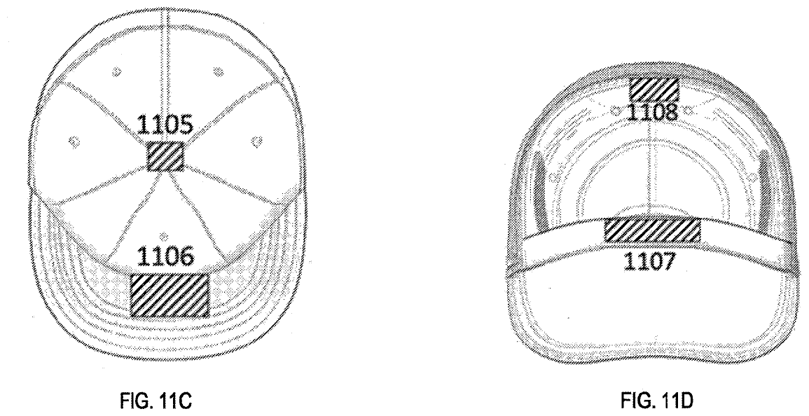

In one embodiment, the CU is integrated to the wearable electronic device. In one exemplary embodiment of the example implementations, the CU is depicted as the striped box in FIGS. 11A-D and it is placed in any of the panels of the crown 1101 or on the visor 1102 in FIG. 11A, or on the button 1103 or closure 1104 in FIG. 11B, or on the bottom of the button 1105 or visor 1106 in FIG. 11C, or on the sweatband 1107 or bottom of the button 1109 in FIG. 11D.

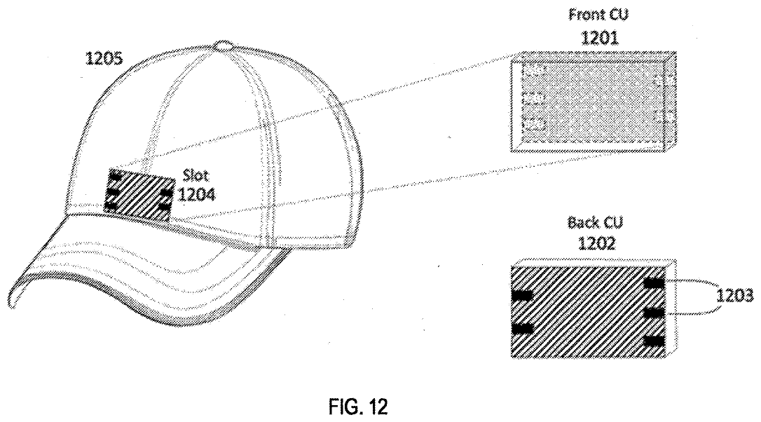

In a different embodiment of the example implementations, the CU is designed in a way that can be removed from the wearable electronic device when not in use. For example, the CU can be mounted on the hat when it's in use, or removed from the hat and placed on the charging station for recharging when not in use. FIG. 12 shows the front side of the CU 1201 and back side of the CU 1202 with five pins 1203 for exemplary purpose, but more in general the CU comprise one or any number of pins with any size or placement within the real estate of the CU. In one embodiment, the pins are made of any conductive material. The wearable electronic device 1205 is equipped with one or a plurality of slots 1204 to host the CU, e.g., when the CU is charged and the wearable is ready to be used. The pins 1203 at the CU match the shape and location of the pins at the slot 1204, so they make contact when the CU is mounted on the slot. The pins implement the connection 1009 in FIG. 10 and are used to connect the CU to the sensors installed or integrated inside the wearable electronic device (e.g., textrodes). We observe that the device 1205 is any of the wearables disclosed above, including any type of hat, glasses, or hair clips. Further, the slot 1204 is placed in one or multiple of the locations shown in FIGS. 11A-D. In another embodiment, the removable CU 1201 is designed to be compatible with a variety of products comprising of the sensors and technology disclosed in this example implementations, including but not limited to: hat, or glasses, or earbuds, or hair clips, or hair band, or headband, or safety helmet, or safety cap, or motorcycle helmet, or racing helmet, or skiing helmet, or climbing hat, or by cycle bump cap, or any other wearable that is worn on someone's head. For example, the user may own one or a plurality of the wearables above, each wearable has integrated sensors and the slot 1204 has the same specifications for all the wearable. The sensors include but are not limited to: EEG electrodes, EMG, fMRI, fNIRS, MEG, PET, EROS, ECG or EKG, PPG, EDA, or antennas that receive or transmit EMF, or 3-axis gyroscope, or accelerometer, or global positioning system (GPS) receiver, or barometer, or body or skin temperature, or sensors for sweat analysis, or proximity sensor, or ambient light sensor, or any sensor to identify the ID of the person. In this embodiment, the user can utilize the same removable CU across all the wearables he owns so that he can buy multiple wearables (e.g., or different types, or same type but different styles) but only needs one CU to operate all of them.

The processor 1003 is implemented as any electronic device that performs any type of computation, such as a central processor unit (CPU), a graphic processing unit (GPU), an application-specific integrated circuit (ASIC), a digital signal processor (DSP), a micro-controller unit (MCU) any single or multi-core platform, any single or multi-threaded processor, any processor implementing virtual machines or Kubernetes containers. In one embodiment, the CU comprises any of the following chipsets: ADS1298 or ADS1299 by Texas instruments [180], sensing module by Entertech [181], TGAT1/TGAM1 or TGAT2 by NeuroSky [182], STM32L053R8 by STMicroelectronics [183]. In one embodiment, the processor receives live data or waveforms from the sensors and implements one or any combination of digital filters, or filterbanks, power amplifier, low-noise amplifier, or digital-to-analog converter (DAC), or analog-to-digital converters (ADC), or DSP algorithms to remove artifacts due to motion or other factors from the waveforms received from the sensors, or dynamic gain control, or DSP methods that process different brainwaves (delta, theta, alpha, mu, beta, or gamma) independently or in combination, or artificial intelligence (AI) or machine learning (ML) methods to process the waveforms in time, frequency or space domains and classify the emotional states or extract other biological or neurological parameters, or any large language model (LLM) method, or any linear algebra methods to compute precoding or post-coding weights to implement the methods disclosed above and in FIGS. 6-7. The DAC and ADC are implemented with 8 bits, 12 bits, 24 bits, or any number of bits and dynamic range.

The memory 1004 is implemented as one or any combination of Random Access Memory (RAM), Read-Only Memory (ROM), Electrically Erasable Programmable Read-Only Memory (EEPROM), Erasable Programmable Read-Only Memory (EPROM), Dynamic Random Access Memory (DRAM), Static Random Access Memory (SRAM), Synchronous Dynamic Random Access Memory (SDRAM), Double Data Rate Synchronous Dynamic Random Access Memory (DDR), Second Generation Double Data Rate Synchronous Dynamic Random Access Memory (DDR2), Third Generation Double Data Rate Synchronous Dynamic Random Access Memory (DDR3), Fourth Generation Double Data Rate Synchronous Dynamic Random Access Memory (DDR4), High Bandwidth Memory (HBM), Magnetoresistive Random Access Memory (MRAM), Phase-change Random Access Memory (PRAM), Non-Volatile Random Access Memory (NVRAM), 3D Crosspoint (3D XPoint). In one embodiment, the memory stored the data, waveforms or parameters provided by the processor.

The power source 1005 provides power to any of the components of the CU and is implemented as any type of battery, including but not limited to: Lithium-ion (Li-ion), Nickel-cadmium (NiCd), Nickel-metal hydride (NiMH), Lead-acid, Alkaline, Zinc-carbon, Lithium-polymer (Li-Po), Silver-oxide, Mercury-oxide, Zinc-air, Sodium-sulfur (NaS), Flow battery, Solid-state battery. In one embodiment, the battery is disposable or rechargeable via any AC adapters, USB chargers, Qi wireless chargers, Portable power banks, Solar chargers, Fast chargers, Trickle chargers, Smart chargers, Inductive chargers, Universal chargers, Multi-port chargers, Battery tender. In a different embodiment, the power source 1005 is any type of power connector or cord that provide energy to the CU such as USB Type-A, USB Type-B, USB Type-C, Lightning connector, MagSafe connector, DC barrel jack, AC power cord, Coaxial power connector, Anderson Powerpole, Speakon connector, IEC 60320 connectors, PowerCON. In another embodiment, the power connector connects to the battery for charging. In another embodiment, the battery is connected to any source of sustainable energy such as solar panels, or windmills installed on any of the panels of the crown 303 or button 308 of the hat, or any other component of the wearable electronic devices in FIGS. 3A-D, 8, 9.

The clock generator 1006 provides the clock reference to the processor 1003 or any other component of the CU. In one embodiment, the clock generator is any of the following types of clocks: Atomic clock, Crystal oscillator, Rubidium clock, GPS disciplined clock, Oven-controlled crystal oscillator (OCXO), Temperature compensated crystal oscillator (TCXO), Voltage-controlled crystal oscillator (VCXO), Phase-locked loop (PLL) based clock, MEMS-based clock.

The I/O mechanism 1007 send or receives data, waveforms or parameters to or from the processor 1003, or memory 1004, or power source 1005, or clock generator 1006, or display 1008, or the software platform 102, 202 in FIGS. 1,2 via the network 103, 203. In one embodiment, the I/O is a network interface card (NIC) or any chipset that implements one or a plurality of wireless or wireline transceivers including but not limited to: Wi-Fi, Bluetooth, NFC, Zigbee, Z-Wave, RFID, Cellular networks (e.g., 2G GSM, 3G WCDMA, HSDPA, 4G LTE, 5G NR, or any 3GPP network), Satellite networks, DSL, Cable modem, Fiber optic, T1/E1, T3/E3, SONET/SDH, ISDN, Ethernet.

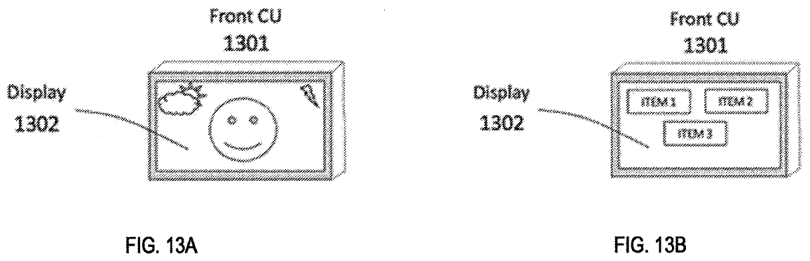

The display 1008 displays any of the data, waveforms, or parameters received from any of the units within the CU or any of the sensors. In one embodiment in FIG. 13A, the display shows any type of image or graph that describes the data, waveforms, or parameters, such as status of the battery charge, weather, emotional states. In another embodiment in FIG. 13B, the display shows information in the form of different menu items that the user can select via buttons or touch screen. In one embodiment, the display changes colors with the changing moods, or affects, or emotional states detected by the wearable electronic devices.

### Software Platform

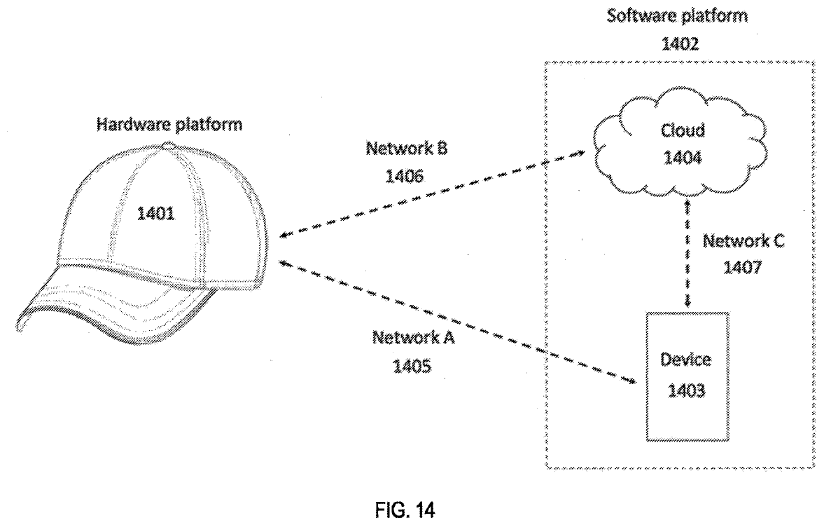

The software platform 1402 in FIG. 14 comprises of one or a plurality of devices 1403 and one or a plurality of cloud infrastructures 1404. The devices 1403 comprise any type of wireline or wireless device including but not limited to: smartphones, tablets, laptops, smartwatches, fitness trackers, wireless headphones, wireless speakers, smart home devices, wireless routers, modems, wireless access points, Wi-Fi extenders, wireless bridges, wireless adapters, wireless cameras, wireless microphones, wireless keyboards, wireless mice, gaming controllers, remote controls.

In one embodiment of the example implementations, the device 1403 runs any of the Apple operating systems (OS) including but not limited to macOS, iOS, iPadOS, watchOS, tvOS, HomePod Software, AudioOS, iPod Software. In another embodiment of the example implementations, the device 1403 runs any of the Android operating systems including but not limited to: Android OS, Android Wear OS, Android TV OS, Android Auto OS, Android Things OS, Fire OS, Oxygen OS, One UI, MIUI, EMUI. In another embodiment, the device 1403 is any type of smartphone or smartwatch devices by any brand included but not limited to: Apple, Samsung, Huawei, Xiaomi, Oppo, Vivo, OnePlus, Google, LG, Sony, HTC, Motorola, Nokia, Asus, Lenovo, ZTE, Meizu, BlackBerry, Alcatel, TCL. In one embodiment, the device 1403 is one or a plurality of smartphone devices, including but not limited to: Apple iphone 13 series (iPhone 13, iPhone 13 mini, iPhone 13 Pro, iphone 13 Pro Max), Samsung Galaxy S21 series (Galaxy S21, Galaxy S21+, Galaxy S21 Ultra), Google Pixel 6 and Pixel 6 Pro, Xiaomi Mi 11 series (Mi 11, Mi 11 Pro, Mi 11 Ultra), OnePlus 9 and OnePlus 9 Pro, Oppo Find X3 series (Find X3 Pro, Find X3 Neo, Find X3 Lite), Vivo X60 series (X60, X60 Pro, X60 Pro+), Motorola Edge 20 series (Edge 20, Edge 20 Pro, Edge 20 Lite), Nokia X20 and X10, Sony Xperia 1 III and Xperia 5 III. In one embodiment, the device 1403 is one or a plurality of smartwatch devices, including but not limited to: Apple Watch Series 1-7, Samsung Galaxy Watch 4 and Watch 4 Classic, Fitbit Versa 1-3, Sense and Sense 2, Garmin Venu 2 and Venu 2S, Fossil Gen 5E and Gen 6, TicWatch Pro 3, Amazfit GTS 2 and GTR 2, Huawei Watch GT 2 Pro, Oppo Watch 2.

The cloud infrastructure 1404 comprise any type of cloud including but not limited to: far-edge cloud, edge-cloud, Public cloud, Private cloud, Hybrid cloud, Community cloud, Distributed cloud, Multi-cloud, Inter-cloud, Fog/cloud edge, Serverless cloud, Amazon Web Services (AWS)—Amazon Elastic Compute Cloud (EC2), Amazon Simple Storage Service (S3), Amazon Relational Database Service (RDS), Microsoft Azure—Azure Virtual Machines, Azure Blob Storage, Azure SQL Database, Google Cloud Platform (GCP)—Compute Engine, Cloud Storage, Cloud SQL, IBM Cloud—Virtual Servers, Object Storage, Databases for MongoDB, Oracle Cloud-Compute, Storage, Database, Alibaba Cloud —Elastic Compute Service (ECS), Object Storage Service (OSS), Relational Database Service (RDS).

The device 1403 is connected to the cloud via a network C 1407. Further, the hardware platform 1401 is the same as disclosed above and in FIGS. 1-13 and it is connected either directly to the device 1403 via a network A, or to the cloud 1404 via a network B, or both. In one exemplary embodiment, the data (i.e., waveforms, brain signals, brainwaves, or any biological parameter) measured at the wearable electronic device 1401 is exchanged directly with the device 1403 over the network A 1405, then the device 1403 exchange some of that data with the cloud 1404, and both device 1403 and cloud 1404 process that data. In another embodiment, the data is exchanged directly between the wearable electronic device 1401 and the cloud 1404, then the cloud exchanges that data with the device 1403, and both device 1403 and cloud 1404 process that data. In other exemplary embodiments, any combination of networks A, B, or C are used to exchange data between the hardware platform 1401, the device 1403 or the cloud 1404, and the data is process by any combination of the hardware platform 1401, the device 1403 or the cloud 1404. The networks A, B or C comprise one or a plurality of wireless or wireline networks including but not limited to: Wi-Fi, Bluetooth, NFC, Zigbee, Z-Wave, RFID, Cellular networks (e.g., 2G GSM, 3G WCDMA, HSDPA, 4G LTE, 5G NR, or any 3GPP network), Satellite networks, DSL, Cable modem, Fiber optic, T1/E1, T3/E3, SONET/SDH, ISDN, Ethernet. The interface for network A or B is implemented in the hardware platform 1401 via the I/O interface 1007 in FIG. 10 and the networks A or B are used to exchange data, waveforms, brainwaves, or parameters between the hardware platform 1401 and the software platform 1402.

In one embodiment, the software platform 1402 is a general purpose platform or is platform agnostic and communicates with any type or number of wearable electronic devices. For example, the software platform 1402 sends or receives data from wearable electronic devices that comprises of any combination of sensors, including but not limited to: EEG electrodes, EMG, fMRI, fNIRS, MEG, PET, EROS, ECG or EKG, PPG, EDA, or antennas that receive or transmit EMF, or 3-axis gyroscope, or accelerometer, or global positioning system (GPS) receiver, or barometer, or body or skin temperature, or sensors for sweat analysis, or proximity sensor, or ambient light sensor, or any ID sensors. The software platform 1402 analyzes data from those sensors and provides users with actionable feedback about physical or mental wellness. In another embodiment, the software platform 1402 communicates via the networks A or B with any hardware platform 1401 including any wearable electronic device, including but not limited to related art commercial devices [4-8] or [17-74].

In one embodiment, the device 1403, or the cloud 1404, or combination of both implement different types of DSP methods to process the data, waveforms or parameters obtained from the hardware platform 1401. In one embodiment, the device 1403 or the cloud 1404 takes data from the accelerometer or gyroscope sensors 1001 through the CU 1002 and the networks A or B, process that data to determine the speed of motion of the user wearing the wearable electronic device and uses that information to remove any artifact of the brainwave signals over time, frequency or space domains. This method is intended to reduce or remove artifacts due to motion or other factors such as interference. In another embodiment, the device 1403 implement methods that select one or a subset of sensors 1001 to be used at any given time. For example, if the sensors are EEG electrodes, the method selects only the electrodes that are in actual contact with the skin and produce a clean brain signal, while discarding all the others. For example, when the EEG electrode is not touching the skin, the voltage of the brain signal is much larger than given threshold, and in that case that signal is recognized and faulty signal and is discarded. In another embodiment, spatial diversity or multiplexing techniques are used across all EEG electrode to enhance the quality of the signal. In another embodiment, the device 1403 calculated precoding weights to implement the methods disclosed in FIGS. 6-7.

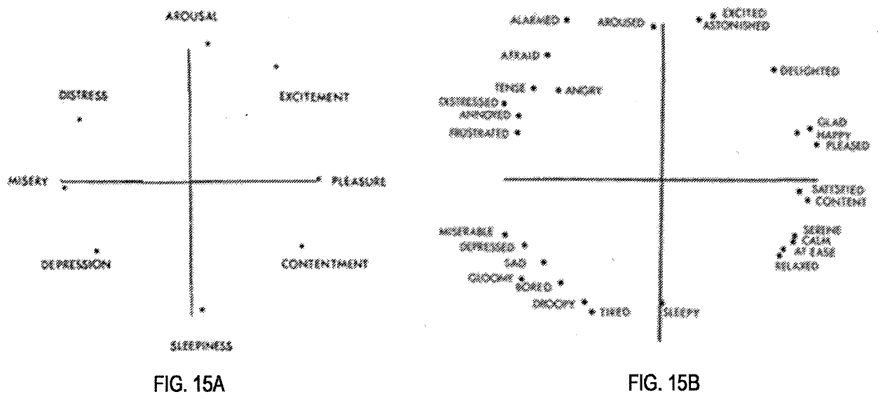

In another embodiment, AI/ML, LLM, or GPT-1, or GPT-2, or GPT-3, or GPT-4 methods are used to analyze brain data for identifying and classifying emotional states. For example, by analyzing brain data in time, frequency and space domains, the device identifies brain signatures that measure different levels of arousal and valence of emotional states. For example, FIGS. 15A-B shows related art circumplex model of affect or emotions [77] depicting over the x-axis different levels of negative/positive valence and over the y-axis different levels of negative/positive arousal. By analyzing brain data, the device computes a given level of valence or arousal and identifies the emotion that best describe the state of the person wearing the wearable electronic device. For example, if high valence and high arousal is detected, then the emotion is labelled as excitement according to the diagram in FIG. 15A. By using FIG. 15B or any other related circumplex model, the device identifies different emotions with finer level of granularity. For example, similarly to the heart rate zones by the Orangetheory [175], the device 1403 analyzes brain data, or heart rate variability, or skin conductance from different sensors 1001 and by combining all that information it computes one or a plurality of mental or physical stress so that the user of the wearable electronic device identifies the level of stress based on different stress zones. The user leverages this information to identify the optimal level of hormetic stress [170] to build emotional resilience and enhance mental wellness.

In one embodiment, one or a plurality of ML libraries are used for implementing the methods, including but not limited to: scikit-learn, TensorFlow, Keras, PyTorch, Theano, Caffe, MXNet, H2O, Spark MLlib, Microsoft Cognitive Toolkit, Torch, Accord.NET, MIpack, Shogun, Weka, CNTK. In one embodiment, one or a plurality of ML libraries for optimization of hyperparameters are used for implementing the methods, Including but not limited to: Scikit-optimize (skopt), Hyperopt, Optuna, SigOpt, Ray Tune, Spearmint, GPyOpt, RoBO, Hyperband, HpBandSter. In one embodiment, the ML algorithms are trained based in brain signals or biological parameters from one or a plurality of users over one or a plurality of data acquisition sessions. In one embodiment, the brain data used to train AI/ML, or LLM, or GPT-1-4 models is first filtered, or reorganized, or grouped in different data sets by feature engineering to extract the information content from the brain data and train the models more efficiently. In one exemplary embodiment with sensors being EEG signals, the brainwave data that ranges between e.g., 0 Hz and 60 Hz is divided into different subbands of given bandwidth, where the bandwidth is chosen based on a desired level of granularity. For example, the band 0-60 Hz is divided into 6 subbands of 10 Hz each, or 12 subbands of 5 Hz each, or 60 subbands of 1 Hz each, and so forth, or any number of subbands with fixed or variable bandwidth across subbands. In another embodiment, the spectrum is devided into the following bands: Delta (1-4 Hz), Theta (4-8 Hz), Alpha-1 (8-10 Hz), Alpha-2 (10-12 Hz), Beta-1 (12-16 Hz), Beta-2 (16-20 Hz), Beta-2 (20-30 Hz), Gamma-1 (30-40 Hz), Gamma-2 (40-50 Hz), Gamma-3 (50-60 Hz). In another embodiment, EEG electrodes are used with qEEG, or BEAM, or LORETA, or sLORETA, or any high-resolution method disclosed in FIGS. 6-7 to compute brain maps, or fNIRS sensors are used to compute the brain maps, then the brain maps are divided into predefined regions, or areas, or volumes according to predetermined grids with different granularities, and the intensity of brain activities in one or a plurality of those regions are used to train the models.

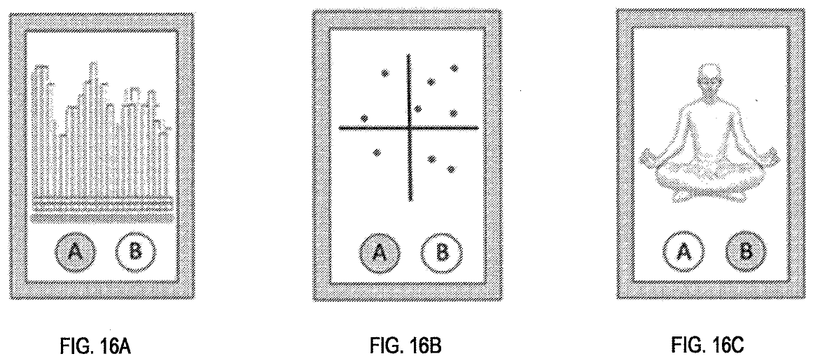

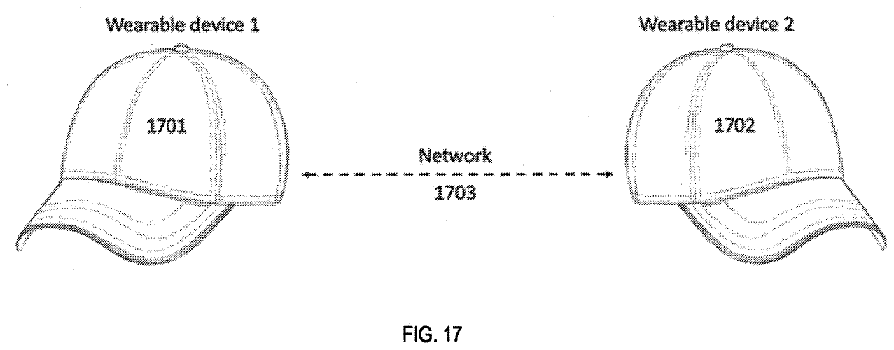

The methods disclosed above are implemented inside an application that runs on the device 1403 or in the cloud 1404. The application comprises of a user experience (UX) and user interface (UI) shown in FIGS. 16A-C. In one exemplary embodiment of the example implementations, the UX/UI includes one or a plurality of tabs the provide different services, such as tabs A and B in FIGS. 16A-C. In one embodiment, tab A is used to display any type of graph depicting any mental or physical performance metrics, including but not limited to: line graph, bar graph, pie chart, scatter plot, area chart, histogram, box plot, bubble chart, radar chart, heat map, waterfall chart, funnel chart, Gantt chart, network diagram, Sankey diagram, spider chart, polar chart. In another embodiment, tab A is used to display the circumplex model of affect or emotions in FIGS. 15A-B to inform the user of their current emotional state. In another embodiment, tab A shows the level of hermetic stress, for example it indicates situations of low, medium or high stress levels and suggests optimal level of stress based on the individual user's data or collection of data from many users available in the cloud 1404. In a different embodiment, tab B shows different training programs to improve the mental or physical performance metrics displayed in tab A, for example, the training programs are in the form of videos, music, video conferencing, implementing different types of activities including but not limited to: Meditation, Yoga, Tai Chi, Qigong, Body Scan, Walking Meditation, Mindful Eating, Mindful Breathing, Mindful Observation, psychotherapy, coaching, program to enhance emotional resilience, neurofeedback-based therapy, neurological regulation, or mental wellness. In another embodiment, the tabs A or B shows the users' personal calendar mapped into their emotional states. In another embodiment, the tabs A or B depict the user's brain maps in real-time obtained by one or a plurality of methods disclosed above, including estimation via fMRI/fNIRS signal processing or via any of the EEG methods above including qEEG, BEAM, LORETA, sLORETA, MUSIC, ESPRIT, Root-MUSIC, MPM, MVDR, BSAP, FBF, SRP, ANN, RBF, SVR, SVM, MLP, DL, DNN.

In another embodiment, the graphs or training programs (e.g., in tab A or B) are used to improve emotional intelligence, which consists of four major pillars including but not limited to: self awareness, self regulation, social awareness and relationship awareness. For example, the graphs and training programs help the users improve any of these pillars to improve their emotional intelligence. The data collected by many users in the cloud 1404 is used to calculate statistical scores (e.g., average stress level in a country) and provide each individual user with that information for example to motivate them to achieve those scores (e.g., national average scores) or at least stay within a range that is considered a healthy score based on psychological research, neurological research, medical advices or data from one of a plurality of users utilizing the software platform 1402.

In another embodiment, the graphs or training programs in FIGS. 16A-C are used to assign different scores to the level of arousal of a given user, for example, low arousal (<5), medium arousal (between 5 and 10), or high arousal (more than 10). For example, too much arousal for prolonged period during the day may desensitize the brain and its reward center, or the brain may get addicted to high level of dopamine and keep craving for more of those arousing experiences (e.g., excessive exposure to video contents showing explicit scenes of sex or violence or horror movies may desensitize the brain and cause mental illness). In one embodiment, the user exploits this information to self regulate their level of arousal and achieve healthy or optimal levels of arousal based on information provided by the device 1403 or the cloud 1404 (e.g., can decided to be exposed to certain video contents only for short period of time or only during certain times of the day to improve mental wellness). Similar method is applied to different levels of valence instead of arousal, or different levels of emotional states as disclosed in FIGS. 15A-B.

In another embodiment, the applications in FIGS. 16A-C defines different levels of hormetic stress [170] and reports different levels of stress including but not limited to: e.g., low stress, medium stress or high stress. When stress level is too low, the application warns the user about the danger to fall into depression or suggests solutions to improve the stress score (e.g., by taking a walk in the nature, or visiting friends, or going to a concert, etc.). When stress level is too high for prolonged periods of time, the application warns the user about imminent possibility of burnout or suggests activities to lower the level of stress (e.g., by taking breaks, or meditating, or breathing exercise, etc.). When the stress level is medium, the application rewards the user to maintain an optimal level of hormetic stress. In one embodiment, the detection of stress, rumination, or depressive states is carried out, for example, by detecting activation of the DMN using e.g., the high-resolution techniques in FIGS. 6-7, or simply by analyzing brain signals from one, or two, or multiple EEG electrodes e.g., in contact with the forehead, or any other sensor layout. For example, the applications in FIGS. 16A-C monitor the level of rumination of the user throughout the day, and when rumination is detected for prolonged periods of time or with high intensity, the applications send alarms to the user to raise awareness about the rumination activity and suggests taking a break, e.g., with meditation or any physical activity. In another embodiment, the applications in FIGS. 16A-C define different tiers of users based on subscription. For example, tier 1 users (e.g., free subscription) get only access to a subset of metrics provided in tab A; tier 2 users (e.g., $5/month subscription) get access to more metrics and key insights on their mental wellness based on those metrics in tab A (e.g., high level stressed, rumination compared to average population); tier 3 users (e.g., $10/month subscription) get access to training programs or videos (e.g., meditation, psychotherapy, coaching) in tab B.

In another embodiment, the application detects symptoms of microstress [191] that manifests as brief and frequent moments of tension in everyday life, which are hard to register and that keep accumulating over time producing high blood pressure, increasing heart rate, triggering hormonal or metabolic changes with resulting increase in body weight that can lead to obesity. The application maps real-time brainwave parameters into microstress (e.g., significant increase in power of beta and gamma waves), and brings awareness to the users about their negative interactions from everyday life that cause microstress, so we can avoid them to improve our overall well-being. For example, the application maps dates and times when microstress was detected to the calendar of the users, so that the users can associate what events throughout their daily life caused microstress warnings.

In another embodiment, the applications FIGS. 16A-C comprise of mental training, medical diagnosis, or commercial applications. Some embodiments of mental training include: monitoring level of focus, concentration, workload, engagement, or fatigue; building emotional resilience (e.g., for athletes, or corporate executives); enhancing emotional and mental awareness (e.g., label emotional states throughout the day, measure emotions); acting training (e.g., Meisner technique and Method acting train actors to experience truthful emotions on set). In another embodiment, the applications are used for medical purposes such as: detecting emotions of infants, patients with neurodegenerative disease, Deaf people; detecting upcoming seizures for people affected by epilepsy; diagnosing strokes, dementia, alcholism, different neurological disorders such as Alzheimer's disease, autism, schizophrenia, major depressive disorder (MDD), chronic pain, post-traumatic stress disorder (PTSD), attention deficit hyperactivity disorder (ADHD), attention deficit disorder (ADD), autism, rumination, or others. In a different embodiment, the applications are for commercial purposes, including but not limited to: marketing, or consumer, or neuroscience research by leveraging cloud-data from multiple users; detecting fatigue for track drivers, airline or general aviation pilots, air traffic controllers, train operators, etc.; augmented reality (AR) or virtual reality (VR) applications (e.g., detecting eye movement with EEG, detecting emotions created by different AR/VR experiences); human activity recognition (HAR) to detect e.g., muscular activities, emotional control, motor planning, emotional expression, reading activities.

In another embodiment, LLM or GPT-1-4 technologies are used to train natural language processing (NLP) methods to map brain signals into thoughts. One exemplary method comprises of the following steps: i) the subject wearing one or a plurality of the wearable electronic devices disclosed above and in FIGS. 1-2 is asked to listen to audio content (e.g., podcast, radio, audiobook, etc.) or video content (music video, film, etc.). The wearable electronic devices are equipped with fNIRS sensors, or EEG electrodes along with qEEG, or BEAM, or LORETA, or sLORETA, or any high-resolution method disclosed in FIGS. 6-7, ii) during this activity, brain maps or brain activities are measured and recorded using the wearable electronic devices; iii) the brain maps and audio/video content are used to train the GPT-1-4 models; iv) after the models are trained, the subject can listen to other audio or video content, or think of different thoughts and the LLM or GPT-1-4 methods decode those thoughts e.g., in text format, based on the brain maps measured in real time by the wearable electronic device. In another embodiment, the wearable electronic device is any pet collar (e.g., for cats or dogs) and the methods are used to decode pet's thoughts into text or audio.

We observe that related art [201] uses brain maps measured with fMRI to decode people's thoughts. As disclosed in [201], that method is limited in that the GPT models must be trained with the person inside the fMRI machines for hours (even 20 hours) before it can be used to decode thoughts. The method in [201] has the limitation that the user needs to be still inside the fMRI machine and needs to intentionally listen to the audio/video content, or else the method would not work. Also, the method has the limitation that the same model does not transfer from person to person, or in other words, if the GPT model is trained on one person, it cannot be transferred to another person. As disclosed in the following transcripts from the podcast in [201]: “not only the training must be done on willing people, what the model learns from those people isn't applicable for anyone but them. Using the model to someone without the training, it says that is doesn't work at all, it's really crummy, statistically detectible as if there is something there, but qualitatively it's garbage”. The reason of this limitation of related art is attributed to the results of the neuroplasticity of our brains: to generate the same thoughts, actions or any activity produced by our brain by different people, each person's brain uses given neuropaths or active brain regions that had developed since their birth over the course of their respective lives, and those neuropaths or active brain regions vary from person to person. Therefore, training GPT models based on spatial activation of different brain regions is fundamentally limiting. By contrast, one of the embodiments of the present example implementations uses the time or frequency dimension of brain data (not the space dimension as related art work) to train GPT models and map brain activities into thoughts. One exemplary embodiment of the example implementations comprises EEG sensors in FIGS. 3A-B to detect brainwaves. Even if the brainwaves associated to a given thought are generated from different brain regions in different persons, the typology of neurotransmitters, or charged particles, or action potentials that generate them is the same across different persons, and as such the time or frequency signatures or brain fingerprints obtained from brainwaves or brain signals is the same. Therefore, the GPT models trained by EEG brain signals on one person are used successfully on any other person to decode their thoughts. Further, because the wearable electronic device is portable, the user does not need to stay still inside an fMRI machine to train the GPT models, rather it can move or perform any activity while the model trains.

In one embodiment of the example implementations, one or multiple wearable electronic devices 1701 and 1702 communicate directly with one another via a network 1703. The network 1703 consists of any wireless or wireline transceivers including but not limited to: Wi-Fi, Bluetooth, NFC, Zigbee, Z-Wave, RFID, Cellular networks (e.g., 2G GSM, 3G WCDMA, HSDPA, 4G LTE, 5G NR, or any 3GPP network), Satellite networks, DSL, Cable modem, Fiber optic, T1/E1, T3/E3, SONET/SDH, ISDN, Ethernet. In another embodiment, the wearable electronic devices communicate via brain-to-brain communications [172], or telepathy, or wirelessly transmitted brainwaves or information between two separate brains. For example, two subjects wearing the wearable electronic devices 1701 and 1702 can communicate with each other without talking and simply by exchanging brain data or their own thoughts through the wearable electronic devices 1701 and 1702. In one exemplary embodiment, the wearable device 1701 measures brain data from the subject wearing it, then utilizes power amplifiers to amplify the analog brain signals, or the CU in FIG. 10 to generate a digital signal, and transmit that analog or digital signals to wearable device 1702, which then receives the analog or digital signals, and focuses the analog or digital signals into one or a plurality of regions inside the brain using the methods above and in FIGS. 6-7 to generate or modulate action potentials at one or multiple frequencies into the neurons, or neurotransmitters, or synapses, in a way that the subject wearing the wearable device 1702 understand or decodes the same thoughts or actions being thought or executed by the subject wearing the wearable device 1701. In another embodiment, thoughts or activities by a first person 1 wearing the wearable electronic device 1701 are converted from analog to digital and digitally decoded by device 1701, then digitally transmitted to the wearable electronic device 1702 over the network 1703, then the device 1702 uses that digital brain data to modulate EMF or brainwaves using high-resolution methods in FIGS. 6-7 and produce action potential to stimulate one specific neuron, or neurotransmitter, or synapses, or groups of neurons, or groups of neurotransmitters, or groups of synapses, with one or multiple frequencies and to stimulate the brain of person 2 wearing the device 1702 to perform the same thoughts or activities as person 1.

In one embodiment of the example implementations, the wearable electronic device is an earpatch 2001 that is placed behind the ear as in FIG. 20. In one embodiment, the earpatch is attached to the skin through any type of adhesive material, or Medical adhesive tape, Silicone gel pads, Double-sided adhesive patches, Hydrocolloid dressings, Skin-friendly adhesives, Medical-grade skin adhesives, Hypoallergenic tapes, Adhesive patches with breathable backing, Adhesive gels, Acrylic-based adhesives, or silicone suction cups, or silicone suction pad, or rubber suction cups or any variation of pads that contain one or a plurality of suction cups.

An exemplary implementation of the earpatch is shown in FIGS. 21A and 21B. In one embodiment of the exemplary implementation, the top side of the earpatch in FIG. 21A hosts the electronics, including one or a plurality of printed circuit boards (PCBs), one or a plurality of battery, or any of the electronics disclosed in FIG. 10. In one embodiment, the PCB is embedded inside soft material to increase comfort of the device while wearing it behind the ear such as: Hydrogel, Silicone gel, Memory foam, Soft fabric, Microfiber, Neoprene, Gel-filled cushions. In another embodiment of the exemplary implementation, the bottom side of the earpatch in FIG. 21B hosts one or a plurality of EEG electrodes that make contact with the skin or hair or scalp to measure one or a plurality of brainwaves. The electrodes in FIG. 21B are connected to the electronics in FIG. 21B. In one embodiment, the earpatch hosts two electrodes, wherein one electrode is the active electrode and the other electrode is the reference electrode, so that the amplitude of the brainwave is the voltage measured between these two electrodes. In one embodiment, the reference electrode is attached to the ear itself to reduce noise of the measured brainwave. In one embodiment, the two electrodes are placed at a distance D, wherein the distance is calculated to maximize the signal quality of the brainwaves measured through the EEG electrodes.

1. An apparatus for a wearable electronic device coupled with a software platform via a network wherein:
the software platform comprises of an application running on a device or in a cloud, or partially on a device and partially on the cloud;
the network comprises of one or a plurality of wireless networks including but not limited to Wi-Fi, Bluetooth, or cellular networks;
the wearable electronic device comprises of a control unit (CU) coupled with one or a plurality of electroencephalogram (EEG) electrodes to measure, analyze or interpret brain or body signals and provide real-time feedback on emotional states including valence or arousal;
the control unit and EEG electrodes are integrated in the wearable electronic device of any regular or irregular shape with soft or spongy material including silicone;
the CU comprises of one or a plurality of processor, memory, power source, clock generator, input/output (I/O) mechanism, display;
the power source is implemented as any type of rechargeable battery.

2. The apparatus in claim 1 wherein the wearable electronic device is placed in one or a plurality of locations within a hat.

3. The apparatus in claim 1 wherein the wearable electronic device is integrated in the frame, lenses or temples of eye glasses.

4. The apparatus in claim 1 wherein the wearable electronic device is one or a plurality of earbuds.

5. The apparatus in claim 1 wherein the EEG electrodes comprise of one or a plurality of textile electrodes (textrodes).

6. An apparatus for a wearable electronic device coupled with a software platform via a network wherein:
the software platform comprises of an application running on a device or in the cloud, or partially on a device and partially on the cloud;
the network comprises of one or a plurality of wireless networks including but not limited to Wi-Fi, Bluetooth, or cellular networks;
the wearable electronic device comprises of a eye glasses including one or a plurality of control units (CUs), and sensors integrated on the bridge, or rim, or temples, or lenses;
the CU comprises of one or a plurality of processor, memory, power source, clock generator, input/output (I/O) mechanism, display;
the sensors comprise of one or a plurality of electroencephalogram (EEG) electrodes to measure, analyze or interpret brain or body signals and provide real-time feedback on emotional states including valence or arousal.

7. The apparatus in claim 6 wherein the eye glasses are used for detecting eye movement with EEG.

8. The apparatus in claim 6 wherein the eye glasses are used for detecting emotions.

9. The apparatus in claim 6 wherein the eye glasses are used for augmented reality (AR) or virtual reality (VR) applications.

10. A method comprising of an artificial intelligence (AI), a machine learning (ML), a large language model (LLM), or a generative pre-trained transformer (GPT) model implemented on a device or a cloud, and used to analyze brain data received from a wearable electronic device equipped with electroencephalogram (EEG) electrodes, for identifying and classifying emotional states or provide neurofeedback therapy based on one or a plurality of EEG data, the method including:
analyzing brain data in time, frequency or space domains;
identifying brain signatures that measure different levels of arousal or valence of emotional states;
computing a given level of valence or arousal and identifying the emotion that best describes the state of the person wearing the wearable electronic device using one or the plurality of AI, ML, LLM or GPT models;
wherein the models are trained based on one or a plurality of brain signals or biological parameters from one or a plurality of users over one or a plurality of data acquisition sessions, and the brain signals are filtered, or reorganized, or grouped in different data sets by feature engineering to extract the information content from the brain data and train the models.

11. The method in claim 10 implemented inside an application that runs on a device, the application comprising of a user experience (UX) and user interface (UI) including one or a plurality of tabs.

12. The UI in claim 11 comprising of one or a plurality of graphs showing one or a plurality of mental or physical performance metrics.

13. The UI in claim 11 comprising of one or a plurality of circumplex model of affect or emotions.

14. The UI in claim 11 comprising of one or a plurality of training programs to improve the mental or physical performance metrics, or emotional intelligence, or self awareness, self regulation, social awareness or relationship awareness.

15. The UI in claim 11 comprising of one or a plurality of training programs to help one or a plurality of users to self regulate their level of arousal and achieve healthy or optimal levels of arousal based on information provided by the device.

16. The UI in claim 11 used for detecting symptoms of microstress, or different levels of hormetic stress, or for to maintaining an optimal level of hormetic stress.

17. The apparatus in claim 1 wherein the wearable electronic device is one or a plurality of earpatches comprising of one or a plurality of printed circuit boards (PCBs) and one or the plurality of EEG electrodes.

18. The apparatus in claim 17 wherein the earpatch is attached to the skin through any type of adhesive material, or silicone suction pad, or silicone or rubber suction cup.

19. The earpatch in claim 17 wherein PCB is embedded in a soft material such as hydrogel.

20. The earpatch in claim 17 wherein distance between EEG electrodes is calculated to maximize the signal quality of the brainwaves measured through the EEG electrodes.

21. An apparatus for a wearable electronic device coupled with a software platform via a network wherein:
the software platform comprises of an application running on a device or in the cloud, or partially on a device and partially on the cloud;
the network comprises of one or a plurality of wireless networks including but not limited to Wi-Fi, Bluetooth, or cellular networks;
the wearable electronic device comprises of a hat including one or a plurality of control units (CUs), and sensors integrated on top of the cloth or integrated within the cloth of the hat;
the CU comprises of one or a plurality of processor, memory, power source, clock generator, input/output (I/O) mechanism, display;
the sensors comprise of one or a plurality of electroencephalogram (EEG) electrodes to measure, analyze or interpret brain or body signals and provide real-time feedback on emotional states including valence or arousal.

22. The apparatus in claim 21 wherein the hat is a baseball cap, or a beanie hat, or a cowboy hat, or any other type or style of hats.

23. The apparatus in claim 21 wherein the EEG electrodes are placed on the front, side or back panels of the crown.

24. The apparatus in claim 21 wherein the plurality of EEG electrodes are an array placed inside the hat and used to create one or a plurality of concurrent volumes in space of electromagnetic field (EMF) or brainwave energy within the brain.

25. An apparatus for a wearable electronic device coupled with a software platform via a network wherein:
the software platform comprises of an application running on a device or in the cloud, or partially on a device and partially on the cloud;
the network comprises of one or a plurality of wireless networks including but not limited to Wi-Fi, Bluetooth, or cellular networks;
the wearable electronic device comprises of an array of EEG electrodes and one or a plurality of control units (CUs);
the CU comprises of one or a plurality of processor, memory, power source, clock generator, input/output (I/O) mechanism, display;
the array of EEG electrodes is used to create one or a plurality of concurrent volumes in space of electromagnetic field (EMF) or brainwave energy within the brain.

26. The apparatus in claim 25 wherein the volumes in space are of any size, e.g., as small as the size or one neuron or even smaller, or as large as the size of the whole brain.

27. The apparatus in claim 25 wherein the size of the volumes in space is inverse proportional to the number of EEG electrodes of the array.

28. The apparatus in claim 25 wherein 10s, or 100s, or 1000s or more EEG electrodes are integrated into the array to reduce the size of the volumes in space for increasing the spatial resolution of the array of EEG electrodes.
